(19) United States
(12) Patent Application Publication
(10) Pub. No.: US 2025/0268800 A1
(43) Pub. Date: Aug. 28, 2025

(54) WET WIPE

(71) Applicant: THE PROCTER & GAMBLE COMPANY, Cincinnati, OH (US)

(72) Inventors: Roy Jerome HARRINGTON, Liberty Township, OH (US); Mackenzie Jean CHILDS, Cincinnati, OH (US); Luis Egberto OQUENDO, Cincinnati, OH (US); Travis Kyle HODGDON, Cincinnati, OH (US); Rebecca Kolakoski KAUFMAN, Cincinnati, OH (US)

(21) Appl. No.: 19/208,651

(22) Filed: May 15, 2025

Related U.S. Application Data
(63) Continuation of application No. PCT/US2023/084731, filed on Dec. 19, 2023.
(60) Provisional application No. 63/434,655, filed on Dec. 22, 2022.

(51) Int. Cl. A61K 8/02; A61K 8/34; A61Q 19/10

(52) U.S. Cl. CPC A61K 8/0204; A61K 8/345; A61Q 19/10; A61K 2800/30; A61K 2800/5426

(57) ABSTRACT

A wet wipe having a liquid composition loaded on a non-woven substrate and methods of using such wet wipes to clean mammals, such as household pets.

### CROSS REFERENCE TO RELATED APPLICATION

This application is a continuation of PCT/US2023/084731 filed on Dec. 19, 2023, which claims the benefit, under 35 U.S.C. § 119(e), of U.S. Provisional Patent Application Ser. No. 63/434,655 filed on Dec. 22, 2022, now expired, the entire disclosures of all of which are fully incorporated by reference herein.

### FIELD OF THE INVENTION

The present disclosure relates to wet wipes comprising a liquid composition loaded on a non-woven substrate and methods of using such wet wipes to clean mammals, such as household pets.

### BACKGROUND

Household pets, such as dogs and cats, are considered to be important members of many homes and families. Household pets are regularly included in family activities and given full access to all areas of family homes, including bedrooms. Pets are also frequently transported in the family automobile. Given the intimate relationships between household pets and their families, pet hygiene is important for families living with household pets.

Regular washing and grooming of a household pet may help to maintain the hygiene of the pet and reduce pet odors. In particular, dirt and debris may adhere to the coats and skin of household pets that regularly go outside, such as dogs. Dogs, in particular, do not have sweat glands in their skin and are not able to sweat, thereby unwanted odors may get trapped on their coats or skin, And, though household pets, such as dogs, can be professionally cleaned and groomed by a pet groomer, professional grooming and cleaning may be costly and does not address an immediate need to clean a dirty pet to avoid tracking dirt into the home. There are disposable, pre-moistened wipes designed for cleaning pets on the market today. However, these wipes often function to dilute and redistribute soils on a pet, without actually collecting and removing the soils, thereby forcing the pet owner to fully bathe the pet, using large amounts of water and soap, and then dry the pet, using towels or a blow dryer, all while keeping the pet contained in the washing area.

There is a need for a convenient and easy-to-use pet cleaning product that can readily clean a dirty pet, without the use of large amounts of water and soap, and prevent the pet from tracking dirt into the home. A cleaning wipe comprising a selected non-woven substrate and a composition comprising a cationic polymer and a high water content would meet this need.

### SUMMARY

Disclosed herein is a wet wipe comprising: (a) a liquid composition comprising: (i) about 0.01% to about 0.2% by weight of the composition of a cationic polymer, where the cationic polymer has a calculated cationic charge density of about 0.1 to about 4.0 and a weight average molecular weight of about 10,000 g/mol to about 1,750,000 g/mol; (ii) from about 0.001 wt. % to about 5 wt. % by weight of the composition of a cleaning solvent selected from the group consisting of C3-C10 diols; (iii) about 95% to about 99.5% by weight of the composition of water; where the composition has a pH of about 3 to about 6; and (b) a non-woven substrate comprising: (i) about 1% to about 90% by weight of the substrate of synthetic fibers; (ii) about 10% to about 100% by weight of the substrate of natural fibers; (iii) where the non-woven substrate has a basis weight from about 40 g/m² to about 100 g/m²; where the loading ratio of liquid composition to non-woven substrate is about 2.0 g/g to about 6.0 g/g.

Also disclosed herein is a wet wipe comprising: (a) a liquid composition comprising: (i) about 0.01% to about 0.2% by weight of the composition of a cationic polymer, where the cationic polymer has a calculated cationic charge density of about 0.1 to about 4.0 and a weight average molecular weight of about 10,000 g/mol to about 1,750,000 g/mol; (ii) about 80% to about 99.5% by weight of the composition of water; where the composition has a surface tension of about 30 mN/m to about 60 mN/m and a pH of about 3 to about 6; and (b) a non-woven substrate comprising: (i) about 1% to about 90% by weight of the substrate of synthetic fibers; (ii) about 10% to about 100% by weight of the substrate of natural fibers; where the non-woven substrate has a basis weight from about 40 g/m2 to about 100 g/m2; where the loading ratio of liquid composition to non-woven substrate is about 2.0 g/g to about 6.0 g/g.

### BRIEF DESCRIPTION OF THE DRAWINGS

FIG. 1 is a schematic illustration of a wet wipe substrate comprising one layer;

FIG. 2 is a schematic illustration of a wet wipe substrate comprising multiple layers;

FIG. 3 is a schematic illustration of a wet wipe substrate comprising a plurality of texture elements that form a pattern; and

FIG. 4 is a schematic illustration of a co-formed wet wipe substrate.

### DETAILED DESCRIPTION

Various non-limiting forms of the present disclosure will now be described to provide an overall understanding of the principles of the structure, function, manufacture, and use of the wet wipes disclosed herein, which comprise a substrate, e.g., a non-woven substrate, and a liquid composition. One or more examples of these non-limiting forms are illustrated in the accompanying drawings. Those of ordinary skill in the art will understand that the wet wipes disclosed herein and illustrated in the accompanying drawings are non-limiting example forms. The features illustrated or described in connection with one non-limiting form may be combined with the features of other non-limiting forms. Such modifications and variations are intended to be included within the scope of the present disclosure.

As used herein, articles such as “a” and “an” when used in a claim, are understood to mean one or more of what is claimed or described.

As used herein, the terms “include”, “includes” and “including” are meant to be non-limiting.

As used herein, “pet” or “household pet” means dogs, cats, small mammals, such as gerbils, hamsters, chinchillas, rats, rabbits, guinea pigs, and ferrets, and/or other domesticated animals.

The term “substantially free of” or “substantially free from” as used herein refers to either the complete absence of an ingredient or a minimal amount thereof merely as impurity or unintended byproduct of another ingredient. A composition that is “substantially free” of/from a component means that the composition comprises less than about 1%, or less than about 0.8%, or less than about 0.5%, or less than about 0.3%, or less than about 0.1%, or less than about 0.05%, or less than about 0.01%, or less than about 0.001%, or about 0% of a component, by weight of the composition.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated. It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As noted above, humans and their household pets, such as dogs, are continuously exposed to and carrying a range of environmental and biological soils. These soils may be tracked through the home and may contribute to the soiling of living spaces in the home. Some environmental soils may even pose health concerns, such as allergies, to both humans and pets. Therefore, an effective and convenient-to-use product and method for collecting and removing a variety of soils, at various transition points throughout a range of daily activities, is needed. An effective and convenient-to-use product enables: pet cleaning to be completed rapidly, particularly during transition to and from the car or home; pet cleaning to be completed using one hand, since the other hand is typically used to hold onto and control the pet; and the removal of soils from various areas of the pet's body on the pet's coat as well its paws. A pet's coat includes the hair or fur on its body.

Current marketed products in the pet personal cleansing wipe category, though marketed as being an effective and an easy-to-use to clean pets (including pet paws), do not meet consumer expectations. For example, some current marketed products comprise lotions that tend to dilute and redistribute soils, rather than removing the soils. The wet wipes disclosed herein address several disadvantages of existing non-rinse cleaning products for cleaning pets, such as wet wipes. Currently marketed wet wipes for pets comprise lotions that may efficiently wet surfaces and soils, but tend to dilute and redistribute the soils on the pet. More specifically, currently marketed wet wipes for pets may suspend soils in a liquid film between the surface of the pet's hair or skin (including on a pet's paws and elsewhere on the pet's body) and the wipe in a diluted state. Once the wipe itself is removed, any excess lotion that contains soil tends to redeposit the soil on the surface of the pet's hair or other nearby surfaces that contact the excess lotion (e.g., user's hands).

The wet wipes disclosed herein address this problem of soil removal, including particulate soil and oily, body soils, which tend to be odorous. The synergistic combination of the selected non-woven substrate and the lotion formulation provides for balanced lotion delivery and soil removal. The liquid composition is formulated to separate different soils from the surface of the pet's hair, while the selected non-woven substrate collects the particulate soils and absorbs oily or hydrophobic odorous soils.

### Liquid Composition/Lotion

The wet wipes disclosed herein comprise an aqueous portion, herein referred to as a liquid composition or lotion. The liquid composition may comprise from about 80% to about 99.5% water, or from about 85% to about 99.5% water, or from about 90% to about 99.5% water, or from about 95% to about 99.5% water, or from about 96% water to about 99.5% water, from about 97% water to about 99.1% water, or from about 98.2% water to about 98.9% water, by weight of the liquid composition, specifically reciting every 0.1% increment within these ranges and any ranges formed therein or thereby, according to the Water Content Method disclosed herein. Overall, a wet wipe liquid composition comprising water within the above-specified ranges may be gentle on a pet's skin, especially when the wet wipe is employed to cleanse delicate areas of a pet's body.

The liquid composition may be substantially free of C1-C4 alkyl monohydric alcohols, such as methanol, ethanol, isopropanol, and butanol, as well as isomers thereof and mixtures thereof. Such monohydric alcohols may be irritating to the pet's skin and/or make the pet ill, if ingested by the pet.

The liquid composition may comprise various other chemical constituents that carry out functions such as wetting, cleaning, buffering, and preservation, as will be discussed in detail herein. The liquid composition disclosed herein is designed to sufficiently wet the surface to be cleaned and to begin transporting soils, including particulate soils and oily, liquid soils, away from the surface of the pet's hair, while balancing the absorbent/non-absorbent properties of the non-woven substrate to effectively collect and remove the mixed soils.

The liquid compositions disclosed herein may be made by the conventional processes described in the art.

The liquid compositions disclosed herein may comprise cationic polymers, particularly selected cationic polymers that aggregate particulate soils, as well as one or more optional, adjunct ingredients.

### Cleaning Solvent

The liquid composition may comprise from about 0.001 wt. % to about 5 wt. %, or from about 0.01 wt. % to about 4 wt. %, or from about 0.05 wt. % to about 2.5 wt. % of one or more cleaning solvents. Tie cleaning solvent may comprise a C3-C10 diol, a C5-C9 diol, a C6-C8 diol, or a mixture thereof. The cleaning solvent may be selected from the group consisting of C3-C10 diols. As used herein, a “C3-C10” diol means a hydrocarbon-based compound comprising two hydroxyl groups and a C3-C10 alkyl group or a C3-C10 alkenyl group, Preferably, the C3-C10 diol is free of ethoxy groups, ester groups, ketone groups, carboxylate groups, or combinations thereof. The C3-C10 diol(s) may be linear or branched, saturated or unsaturated, cyclic or acyclic, Preferably, the C3-C10 diol(s) are linear, saturated, and acyclic. The C3-C10 diol nay consist essentially of two hydroxyl groups and from three to ten —CH₂— groups. Without wishing to be bound by theory, it is believed that a cleaning solvent comprising two hydroxyl groups and three to ten carbon atoms is sufficiently amphiphilic to reduce surface tension and help break up oils and dirt on skin and fur, without being so amphiphilic that it emulsifies, stabilizes, or otherwise suspends the oil and dirt in the liquid composition.

Suitable C3-C10 diols include propanediol, butanediol, pentanediol, hexanediol, benzenediol, heptanediol, octanediol, nonanediol, decanediol, isomers thereof, and mixtures thereof. Suitable C3-C10 diols include 1,1-propanediol, 1,2-propanediol, 1,3-propanediol, 1,1-butanediol, 1,2-butanediol, 1,3-butanediol, 1,4-butanediol, 2,3-butanediol, 1,1-pentanediol, 1,2-pentanediol, 1,3-pentanediol, 1,4-pentanediol, 1,5-pentanediol, 2,4-pentanediol, 1,1-hexanediol, 1,2-hexanediol, 1,3-hexanediol, 1,4-hexanediol, 1,5-hexanediol, 1,6-hexanediol, 2-methyl-2,4-pentanediol, benzene-1,2-diol, benzene-1,3-diol, benzene-1,4-diol, 1,2-heptanediol, 1,3-heptanediol, 1,4-heptanediol, 1,5-heptanediol, 1,6-heptanediol, 1,7-heptanediol, 2,3-heptanediol, 2,4-heptanediol, 2,6-heptanediol, 3,4-heptanediol, 3,6-heptanediol, 1,2-octanediol (caprylyl glycol), 1,3-octanediol, 1,4-octanediol, 1,8-octanediol, 2,3-octanediol, 2,4-octanediol, 2,5-octanediol 2,6-octanediol, 4,5-octanediol, 2-ethyl-1,3-hexanediol, 1,2-nonanediol, 1,3-nonanediol, 1,4-nonanediol, 1,7-nonanediol, 1,8-nonanediol, 2,5-nonanediol, 3,6-nonanediol, 4,5-nonanediol, 2,6-dimethyl-3,5-heptanediol, 1,1-decanediol, 1,2-decanediol, 1,4-decanediol, 1,5-decanediol, 1,10-decanediol, 2,4-decanediol, 4,7-decanediol, 5,6-decanediol, and mixtures thereof.

Suitable C5-C9 diols include 1,1-pentanediol, 1,2-pentanediol, 1,3-pentanediol, 1,4-pentanediol, 1,5-pentanediol, 2,4-pentanediol, 1,1-hexanediol, 1,2-hexanediol, 1,3-hexanediol, 1,4-hexanediol, 1,5-hexanediol, 1,6-hexanediol, 2-methyl-2,4-pentanediol, benzene-1,2-diol, benzene-1,3-diol, benzene-1,4-diol, 1,2-heptanediol, 1,3-heptanediol, 1,4-heptanediol, 1,5-heptanediol, 1,6-hepanediol, 1,7-heptanediol, 2,3-heptanediol, 2,4-heptanediol, 2,6-heptanediol, 3,4-heptanediol, 3,6-heptanediol, 1,2-octanediol (caprylyl glycol), 1,3-octanediol, 1,4-octanediol, 1,8-octanediol, 2,3-octanediol, 2,4-octanediol, 2,5-octanediol 2,6-octanediol, 4,5-octanediol, 2-ethyl-1,3-hexanediol, 1,2-nonanediol, 1,3-nonanediol, 1,4-nonanediol, 1,7-nonanediol, 1,8-nonanediol, 2,5-nonanediol, 3,6-nonanediol, 4,5-nonanediol, 2,6-dimethyl-3,5-heptanediol, and mixtures thereof.

Suitable C6-C8 diols include 1,1-hexanediol, 1,2-hexanediol, 1,3-hexanediol, 1,4-hexanediol, 1,5-hexanediol, 1,6-hexanediol, 2-methyl-2,4-pentanediol, benzene-1,2-diol, benzene-1,3-diol, benzene-1,4-diol, 1,2-heptanediol, 1,3-heptanediol, 1,4-heptanediol, 1,5-heptanediol, 1,6-hepanediol, 1,7-heptanediol, 2,3-heptanediol, 2,4-heptanediol, 2,6-heptanediol, 3,4-heptanediol, 3,6-heptanediol, 1,2-octanediol (caprylyl glycol), 1,3-octanediol, 1,4-octanediol, 1,8-octanediol, 2,3-octanediol, 2,4-octanediol, 2,5-octanediol 2,6-octanediol, 4,5-octanediol, 2-ethyl-1,3-hexanediol, and mixtures thereof.

The cleaning solvent may be a selected from the group consisting of 1,2-hexanediol, 1,2-octanediol (caprylyl glycol), and mixtures thereof. The cleaning solvent may comprise a mixture of 1,2-hexanediol and 1,2-octanediol (or caprylyl glycol) (commercially available as SYMDIOL® 68 from Symrise, AG. Branchburg, N.J.).

### Cationic Polymer

The liquid compositions of the present disclosure may comprise a cationic polymer (or mixtures of cationic polymers). The liquid compositions may comprise from about 0.01% to about 1.0% of a cationic polymer, or from about 0.01% to about 0.5% of a cationic polymer, or from about 0.01% to about 0.4% of a cationic polymer, or from about 0.01% to about 0.3% of a cationic polymer, or from about 0.01% to about 0.2% of a cationic polymer, by weight of the liquid composition, specifically reciting every 0.01% increment within these ranges and any ranges formed therein. Suitable cationic polymers will have cationic charge densities of about 0.05 meq/gm, or about 0.1 meq/gm, or about 0.5 meq/gm to about 5.0 meq/gm, or to about 4.0 meq/gm, or to about 3.0 meq/gm, or to about 2.0 meq/gm, at the pH of intended use of the composition, which pH will generally range from about pH 3 to about pH 8, or about pH 3 to about pH 6. As used herein, the term “calculated cationic charge density” (CCCD) means the amount of net positive charge present per gram of the polymer. CCCD (in units of equivalents of charge per gram of polymer) may be calculated according to the following equation:

$$\frac{CCCD = ( Qc \times mol \% c ) - ( Qa \times mol \% a )}{( mol \% c \times MWc ) + ( mol \% n \times MWn ) + ( mol \% a \times MWa )}$$

where: Qc and Qa are the molar equivalents of charge of the cationic, nonionic, and anionic repeat units (if any), respectively; mol % c, mol % n, and mol % a are the molar ratios of the cationic, nonionic, and anionic repeat units (if any), respectively; and MWc, MWn, and MWa are the molecular weights of the cationic, nonionic, and anionic repeat units (if any), respectively. To convert equivalents of charge per gram to milliequivalents of charge per gram (meq/g), multiply equivalents by 1000. If a polymer comprises multiple types of cationic repeat units, multiple types of nonionic repeat units, and/or multiple types of anionic repeat units, the equation can be adjusted accordingly. As used herein “mol %” refers to the relative molar percentage of a particular monomeric structural unit in a polymer. It is understood that within the meaning of the present disclosure, the relative molar percentages of all monomeric structural units that are present in the cationic polymer add up to 100 mol %.

By way of example, a cationic homopolymer (molar ratio=100% or 1.00) having a monomer molecular weight of 161.67 g/mol, the CCCD is calculated as follows: polymer cationic charge density is [(1)×(1.00)/(161.67)]×1000=6.19 meq/g. A copolymer having a cationic monomer with a molecular weight of 161.67 g/mol and a neutral co-monomer having a molecular weight of 71.079 g/mol in a mol ratio of 1:1 is calculated as (1×0.50)/[(0.50×161.67)+(0.50×71.079)]×1000=4.3 meq/g. A terpolymer having a cationic monomer having a molecular weight of 161.67, a neutral co-monomer having a molecular weight of 71.079 g/mol, and an anionic co-monomer having a neutralized molecular weight of 94.04 g/mol in a mol ratio of 20:75:5 has a CCCD of 1.7 meq/g.

The weight average molecular weight of such suitable cationic polymers will generally be about 10,000 g/mol to about 3,000,000 g/mol, or about 10,000 g/mol to about 2,000,000 g/mol, or about 10,000 g/mol to about 1,750,000 g/mol. As used herein, the term “molecular weight” refers to the weight-average molecular weight of the polymer chains in a polymer composition. Further, as used herein, the “weight-average molecular weight” (“Mw”) is calculated using the equation:

$$Mw = \frac{( \sum i Ni {Mi}^{2} )}{( \sum i Ni Mi )}$$

where Ni is the number of molecules having a molecular weight Mi.

The cationic polymer may have molar ratio of cationic or pH dependent chargeable monomers to non-cationic or non-chargeable monomers of from about 1:99 to about 20:80. The cationic polymer may be a synthetic polymer, a naturally derived polymer, or a naturally derived and then modified polymer. Examples of naturally derived polymers or naturally derived and then modified polymers include cationic peptides and proteins, as well as polysaccharides such as cationic guar, cationic chitosan, cationic dextran, cationic cellulose, cationic cyclodextrin, cationic starch, cationic pectin, cationic polyglucan, and their derivatives.

The cationic polymers may have at least one positively charged and/or pH dependent chargeable moiety that is at least one of a quaternary ammonium group, a primary amino group, a secondary amino group, or a tertiary amino group. A pH dependent chargeable primary amino group, secondary amino group, or tertiary amino group becomes predominantly positively charged when dissolved in an aqueous medium having a pH less than the pKa of the protonated primary amino group, secondary amino group, or tertiary amino group according to the Henderson-Hasselbach equation:

$$pH = pKa + {log}_{10} ( [ A ] / [ {HA}^{+} ]$$

pKa=−log ₁₀Ka,
Ka is the dissociation constant of the reaction HA⁺+H₂O→A+H₃O⁺,
[A]=the concentration of a primary, secondary, or tertiary amine, and
[HA⁺]=the concentration of a protonated primary, secondary, or tertiary amine. Suitable cationic polymers may include at least one of (a) a cationic guar polymer, (b) a cationic non-guar galactomannan polymer, (c) a synthetic, non-crosslinked, cationic polymer, and/or (d) a cationic cellulose polymer. Additionally, the cationic polymer may be a mixture of cationic polymers.

Synthetic cationic polymers may be made by a wide variety of techniques, including bulk, solution, emulsion, or suspension polymerization. Polymerization methods and techniques for polymerization are described generally in Encyclopedia of Polymer Science and Technology, Interscience Publishers (New York), Vol. 7, pp. 361-431 (1967), and Kirk-Othmer Encyclopedia of Chemical Technology, 3rd edition, Vol 18, pp. 740-744, John Wiley & Sons (New York), 1982, both incorporated by reference herein. See also Sorenson, W. P. and Campbell, T. W., Preparative Methods of Polymer Chemistry. 2nd edition, Interscience Publishers (New York), 1968, pp. 248-251, incorporated by reference herein, for general reaction techniques suitable for the present invention. In one example, the polymers are made by free radical copolymerization, using water soluble initiators. Suitable free radical initiators include, but are not limited to, thermal initiators, redox couples, and photochemical initiators. Redox and photochemical initiators may be used for polymerization processes initiated at temperatures below about 30° C. (86° F.). Such initiators are described generally in Kirk-Othmer Encyclopedia of Chemical Technology, 3rd edition, John Wiley & Sons (New York), Vol. 13, pp. 355-373 (1981), incorporated by reference herein. Typical water soluble initiators that can provide radicals at 30° C. or below include redox couples, such as potassium persulfate/silver nitrate, and ascorbic acid/hydrogen peroxide. In some examples, the method utilizes thermal initiators in polymerization processes conducted above 40° C. (104° F.). Water soluble initiators that can provide radicals at 40° C. (104° F.) or higher can be used. These include, but are not limited to, hydrogen peroxide, ammonium persulfate, and 2,2′-azobis(2-amidinopropane) dihydrochloride. In one example, water soluble starting monomers are polymerized in an aqueous alcohol solvent at 60° C. (140° F.) using 2,2′-azobis(2-amidinopropane) dihydrochloride as the initiator.

A synthetic cationic polymer may include several different monomeric units and be referred to as a copolymer (versus a homopolymer, which includes a single type of monomeric unit). An example of a cationic homopolymer includes polyethylenimine. The cationic polymers of the present disclosure may be random copolymers. The cationic polymers of the present disclosure may be water-soluble and/or water-dispersible, meaning that the polymer does not, over at least a certain pH and concentration range, form a two-phase composition in water at 23° C.±2.2° C. The cationic polymers of the present disclosure may comprise monomeric units such as those described below.

The cationic polymer may be a polyquaternium polymer or blend of two or more polyquaternium polymers. Examples of suitable polyquaternium polymers include Polyquaternium-2 (Poly[bis(2-chloroethyl) ether-alt-1,3-bis[3-(dimethylamino)propyl]urea]), Polyquaternium-4 (Hydroxyethyl cellulose dimethyl diallylammonium chloride copolymer; Diallyldimethylammonium chloride-hydroxyethyl cellulose copolymer), Polyquaternium-5 (Copolymer of acrylamide and quaternized dimethylammoniumethyl methacrylate), Polyquaternium-7 (Copolymer of acrylamide and diallyldimethylammonium chloride), Polyquaternium-8 (Copolymer of methyl and stearyl dimethylaminoethyl ester of methacrylic acid, quaternized with dimethylsulphate), Polyquaternium-10 (Quaternized hydroxyethyl cellulose), Polyquaternium-11 (Copolymer of vinylpyrrolidone and quaternized dimethylaminoethyl methacrylate), Polyquaternium-12 (Ethyl methacrylate/abietyl methacrylate/diethylaminoethyl methacrylate copolymer quaternized with dimethyl sulfate), Polyquaternium-13 (Ethyl methacrylate/oleyl methacrylate/diethylaminoethyl methacrylate copolymer quaternized with dimethyl sulfate), Polyquaternium-15 (Acrylamide-dimethylaminoethyl methacrylate methyl chloride copolymer), Polyquaternium-16 (Copolymer of vinylpyrrolidone and quaternized vinylimidazole), Polyquaternium-17 (Adipic acid, dimethylaminopropylamine and dichloroethylether copolymer), Polyquaternium-18 (Azelaic acid, dimethylaminopropylamine and dichloroethylether copolymer), Polyquaternium-19 (Copolymer of polyvinyl alcohol and 2,3-epoxypropylamine), Polyquaternium-20 (Copolymer of polyvinyl octadecyl ether and 2,3-epoxypropylamine), Polyquaternium-22 (Copolymer of acrylic acid and diallyldimethylammonium Chloride), Polyquaternium-24 (Quaternary ammonium salt of hydroxyethyl cellulose reacted with a lauryl dimethyl ammonium substituted epoxide), Polyquaternium-27 (Block copolymer of Polyquaternium-2 and Polyquaternium-17), Polyquaternium-28 (Copolymer of vinylpyrrolidone and methacrylamidopropyl trimethylammonium), Polyquaternium-29 (Chitosan modified with propylen oxide and quaternized with epichlorhydrin), Polyquaternium-30 (Ethanaminium, N-(carboxymethyl)-N,N-dimethyl-2-[(2-methyl-1-oxo-2-propen-1-yl)oxy]-, inner salt, polymer with methyl 2-methyl-2-propenoate), Polyquaternium-32 (Poly(acrylamide 2-methacryloxyethyltrimethyl ammonium chloride)), Polyquaternium-33 (Copolymer of trimethylaminoethylacrylate salt and acrylamide), Polyquaternium-34 (Copolymer of 1,3-dibromopropane and N,N-diethyl-N′,N′-dimethyl-1,3-propanediamine), Polyquaternium-35 (Methosulphate of the copolymer of methacryloyloxyethyltrimethylammonium and of methacryloyloxyethyldimethylacetylammonium), Polyquaternium-36 (Copolymer of N,N-dimethylaminoethylmethacrylate and buthylmethacrylate, quaternized with dimethylsulphate), Polyquaternium-39 (Terpolymer of acrylic acid, acrylamide and diallyldimethylammonium Chloride), Polyquaternium-43 (Copolymer of acrylamide, acrylamidopropyltrimonium chloride, 2-amidopropylacrylamide sulfonate and dimethylaminopropylamine), Polyquaternium-44 (3-Methyl-1-vinylimidazolium methyl sulfate-N-vinylpyrrolidone copolymer), Polyquaternium-45 (Copolymer of (N-methyl-N-ethoxyglycine)methacrylate and N,N-dimethylaminoethylmethacrylate, quaternized with dimethyl sulphate), Polyquaternium-46 (Terpolymer of vinylcaprolactam, vinylpyrrolidone, and quaternized vinylimidazole), and Polyquaternium-47 (Terpolymer of acrylic acid, methacrylamidopropyl trimethylammonium chloride, and methyl acrylate).

Nonionic Monomeric Units Nonionic monomeric units may be at least one of: nonionic hydrophilic monomeric units, nonionic hydrophobic monomeric units, or mixtures thereof. Non-limiting examples of nonionic hydrophilic monomeric units suitable for the present invention include nonionic hydrophilic monomeric units derived from nonionic hydrophilic monomers that are at least one of: hydroxyalkyl esters of α,β-ethylenically unsaturated acids, such as hydroxyethyl or hydroxypropyl acrylates and methacrylates, glyceryl monomethacrylate, α,β-ethylenically unsaturated amides such as acrylamide, N,N-dimethylacrylamide, N,N-dimethylmethacrylamide, N-methylolacrylamide, a, O-ethylenically unsaturated monomers bearing a water-soluble polyoxyalkylene segment of the poly(ethylene oxide) type, such as poly(ethylene oxide) α-methacrylates (Bisomer S20W, S10W, etc., from Laporte) or α,ω-dimethacrylates, Sipomer BEM from Rhodia (ω-behenyl polyoxyethylene methacrylate), Sipomer SEM-25 from Rhodia (ω-tristyrylphenyl polyoxyethylene methacrylate), α,β-ethylenically unsaturated monomers, which are precursors of hydrophilic units or segments, such as vinyl acetate, which, once polymerized, can be hydrolyzed in order to give rise to vinyl alcohol units or polyvinyl alcohol segments, vinylpyrrolidones, α,β-ethylenically unsaturated monomers of the ureido type, and in particular 2-imidazolidinone-ethyl methacrylamide (Sipomer WAM II from Rhodia), or mixtures thereof. The nonionic hydrophilic monomeric unit may be derived from acrylamide. Non-limiting examples of nonionic hydrophobic monomeric units suitable for the present disclosure include nonionic hydrophobic monomeric units derived from nonionic hydrophobic monomers that are at least one of: vinylaromatic monomers such as styrene, alpha-methylstyrene, vinyltoluene, vinyl halides or vinylidene halides, such as vinyl chloride, vinylidene chloride, C₁-C₁₂ alkylesters of α,β-monoethylenically unsaturated acids such as methyl, ethyl or butyl acrylates and methacrylates, 2-ethylhexyl acrylate, vinyl esters or allyl esters of saturated carboxylic acids, such as vinyl or allyl acetates, propionates, versatates, stearates, α,β-monoethylenically unsaturated nitriles containing from 3 to 12 carbon atoms, such as acrylonitrile, methacrylonitrile, α-olefins such as ethylene, conjugated dienes, such as butadiene, isoprene, chloroprene, or mixtures thereof.

Cationic Monomeric Units Non-limiting examples of suitable cationic or pH dependent chargeable monomeric units include amine containing monomeric units derived from monomers that are at least one of: N,N-(dialkylamino-ω-alkyl)amides of α,β-monoethylenically unsaturated carboxylic acids, such as N,N-dimethylaminomethyl-acrylamide or -methacrylamide, 2-(N,N-dimethylamino)ethylacrylamide or -methacrylamide, 3-(N,N-dimethylamino)propylacrylamide or -methacrylamide, and 4-(N,N-dimethylamino)butylacrylamide or -methacrylamide, α,β-monoethylenically unsaturated amino esters such as 2-(dimethylamino)ethyl acrylate (DMAA), 2-(dimethylamino)ethyl methacrylate (DMAM), 3-(dimethylamino)propyl methacrylate, 2-(tert-butylamino)ethyl methacrylate, 2-(dipentylamino)ethyl methacrylate, and 2(diethylamino)ethyl methacrylate, vinylpyridines, vinylamine, vinylimidazolines, monomers that are precursors of amine functions such as N-vinylformamide, N-vinylacetamide, which give rise to primary amine functions by simple acid or base hydrolysis, acryloyl- or acryloyloxyammonium monomers such as trimethylammonium propyl methacrylate chloride, trimethylammonium ethylacrylamide or -methacrylamide chloride or bromide, trimethylammonium butylacrylamide or -methacrylamide methyl sulfate, trimethylammonium propylmethacrylamide methyl sulfate, (3-methacrylamidopropyl)trimethylammonium chloride (MAPTAC), (3-methacrylamidopropyl)trimethylammonium methyl sulphate (MAPTA-MES), (3-acrylamidopropyl)trimethylammonium chloride (APTAC), methacryloyloxyethyl-trimethylammonium chloride (METAC) or methyl sulfate, and acryloyloxyethyltrimethylammonium chloride (AETAC); 1-ethyl-2-vinylpyridinium or 1-ethyl-4-vinylpyridinium bromide, chloride or methyl sulfate; N,N-dialkyldiallylamine monomers such as N,N-dimethyldiallylammonium chloride (DADMAC); polyquaternary monomers such as dimethylaminopropylmethacrylamide chloride and N-(3-chloro-2-hydroxypropyl)trimethylammonium (DIQUAT or DQ) and 2-hydroxy-N1-(3-(2((3-methacrylamidopropyl)dimethylammino)-acetamido)propyl)-N1, N1, N3, N3, N3-pentamethylpropane-1,3-diaminium chloride (TRIQUAT or TQ), or mixtures thereof. In one example, the cationic monomeric unit comprises a quaternary ammonium monomeric unit, for example a monoquaternary ammonium monomeric unit, a diquaternary ammonium monomeric unit and a triquaternary monomeric unit. In some examples, the cationic monomeric unit is derived from MAPTAC. In some examples, the cationic monomeric unit may be derived from DADMAC. In some examples, the cationic monomeric unit may be derived from TQ. In some examples, the non-ionic monomers are selected from acrylamide derivatives from the group consisting of: acrylamide, mono-alkyl substituted acrylamide, symmetrical or asymmetrical, di-N-alkyl substituted acrylamide derivatives, methacrylamide, mono-alkyl substituted methacrylamide, symmetrical or asymmetrical, di-N-alkyl substituted methacrylamide derivatives and mixtures thereof. In some examples, the acrylamide derivatives of the present disclosure are at least one of: N,N-dimethylacrylamide (NDMAAM), acrylamide, methyl acrylamide, ethylacrylamide, N,N-diethylacrylamide, methacrylamide, N,N-dimethyl methacrylamide, or mixtures thereof.

Further examples of suitable cationic monomeric units include cationic or pH-dependent chargeable monomeric units that are at least one of: N,N-(dialkylamino-ω-alkyl)amides of α,β-monoethylenically unsaturated carboxylic acids, such as N,N-dimethylaminomethylacrylamide or -methacrylamide, 2-(N,N-dimethylamino)ethylacrylamide or -methacrylamide, 3-(N,N-dimethylamino)propylacrylamide or -methacrylamide, and 4-(N,N-dimethylamino)butylacrylamide or -methacrylamide, α,β-monoethylenically unsaturated amino esters such as 2-(dimethylamino)ethyl acrylate (DMAA), 2-(dimethylamino)ethyl methacrylate (DMAM), 3-(dimethylamino)propyl methacrylate, 2-(tert-butylamino)ethyl methacrylate, 2-(dipentylamino)ethyl methacrylate, and 2(diethylamino)ethyl methacrylate, vinylpyridines, vinylamine, vinylimidazolines, monomers that are precursors of amine functions such as N-vinylformamide, N-vinylacetamide, which give rise to primary amine functions by simple acid or base hydrolysis, acryloyl- or acryloyloxyammonium monomers such as trimethylammonium propyl methacrylate chloride, trimethylammonium ethylacrylamide or -methacrylamide chloride or bromide, trimethylammonium butylacrylamide or -methacrylamide methyl sulfate, trimethylammonium propylmethacrylamide methyl sulfate, (3-methacrylamidopropyl)trimethylammonium chloride (MAPTAC), (3-methacrylamidopropyl)trimethylammonium methyl sulphate (MAPTA-MES), (3-acrylamidopropyl)trimethylammonium chloride (APTAC), methacryloyloxyethyl-trimethylammonium chloride or methyl sulfate, and acryloyloxyethyltrimethylammonium chloride; 1-ethyl-2-vinylpyridinium or 1-ethyl-4-vinylpyridinium bromide, chloride or methyl sulfate; N,N-dialkyldiallylamine monomers such as N,N-dimethyldiallylammonium chloride (DADMAC); polyquaternary monomers such as dimethylaminopropylmethacrylamide chloride and N-(3-chloro-2-hydroxypropyl)trimethylammonium (DIQUAT or DQ) and 2-hydroxy-N¹-(3-(2((3-methacrylamidopropyl)dimethylammino)-acetamido)propyl)-N¹, N¹, N³, N³, N³-pentamethylpropane-1,3-diaminium chloride (TRIQUAT or TQ), or mixtures thereof. In some examples, the cationic monomeric unit comprises a quaternary ammonium monomeric unit, for example a monoquaternary ammonium monomeric unit, a diquaternary ammonium monomeric unit and a triquaternary monomeric unit. In some examples, the cationic monomeric unit is derived from MAPTAC. In some examples, the cationic monomeric unit is derived from DADMAC. In some examples, the cationic monomeric unit is derived from TQ. Suitable pH-dependent chargeable monomeric units include dimethylaminoethyl (meth)acrylate, dimethylaminopropyl (meth)acrylate, di-tert-butylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine and vinyl imidazole, and mixtures thereof.

In some examples, the cationic monomeric units are at least one of: trimethylammonium ethyl (meth)acrylate bromide, chloride or methyl sulfate, trimethylammonium ethyl (meth)acrylate bromide, chloride or methyl sulfate, trimethylammonium ethyl (meth)acrylate bromide, chloride or methyl sulfate, dimethylaminoethyl (meth)acrylate benzyl chloride, 4-benzoylbenzyl dimethylammoniumethyl (meth)acrylate bromide, chloride or methyl sulfate, trimethylammonium ethyl (meth)acrylamido bromide, chloride, or methyl sulfate, trimethylammonium propyl (meth)acrylamido braomide, chloride, or methyl sulfate, vinyl benzyl trimethyl ammonium bromide, chloride or methyl sulfate, diallyldimethyl ammonium chloride, 1-ethyl-2-vinylpyridinium bromide, chloride or methyl sulfate, 4-vinylpyridinium bromide, chloride or methyl sulfate, and mixtures thereof.

The liquid composition may comprise a cationic guar polymer—a cationically substituted guar gum derivative. Guar gum for use in preparing these guar gum derivatives is typically obtained as a naturally occurring material from the seeds of the guar plant. The guar molecule itself is a straight chain mannan, which is branched at regular intervals with single membered galactose units on alternative mannose units. The mannose units are linked to each other by means of b(1-4) glycosidic linkages. The galactose branching arises by way of an a(1-6) linkage. Cationic derivatives of the guar gums are obtained by reaction between the hydroxyl groups of the polygalactomannan and reactive quaternary ammonium compounds. The degree of substitution of the cationic groups onto the guar structure is selected to be sufficient to provide the preferred charge density range described above. Suitable guar polymers may have a weight average molecular weight of less than about 10,000,000 g/mol, or from about 400,000 g/mol to about 10,000,000 g/mol, or from about 500,000 g/mol to about 5 million g/mol, or from about 750,000 g/mol to about 3,000,000 g/mol, or from about 1,000,000 to about 2,000,000 g/mol. The cationic guar polymer may have a charge density of from about 0.4 to about 4.0 meq/g, or from about 0.6 to about 3.0 meq/g, or from about 0.75 to about 2.5 meq/g; or from about 1.0 meq/g to about 2.0 meq/g.

Suitable cationic guar polymers include cationic guar gum derivatives, such as guar hydroxypropyltrimonium chloride. The cationic guar polymer may be a guar hydroxypropyltrimonium chloride. Specific examples of guar hydroxypropyltrimonium chlorides include the Jaguar® series commercially available from Solvay (Solvay USA Inc., Cincinnati, OH), for example Jaguar® C-500, commercially available from Solvay. Jaguar® C-500 has a charge density of 0.8 meq/g and a molecular weight of 500,000 g/mol. Other suitable guar hydroxypropyltrimonium chloride are: guar hydroxypropyltrimonium chloride which has a charge density of about 1.3 meq/g and a molecular weight of about 500,000 g/mol and is available from Solvay as Jaguar® Optima. Other suitable guar hydroxypropyltrimonium chloride are: guar hydroxypropyltrimonium chloride which has a charge density of about 0.7 meq/g and a molecular weight of about 1,500,000 g/mol and is available from Solvay as Jaguar® Excel. Other suitable guar hydroxypropyltrimonium chloride are: guar hydroxypropyltrimonium chloride which has a charge density of about 1.1 meq/g and a molecular weight of about 500,000 g/mol and is available from ASI. Other suitable guar hydroxypropyltrimonium chloride are: Hi-Care 1000, which has a charge density of about 0.7 meq/g and a molecular weight of about 600,000 g/mole and is available from Solvay; N-Hance 3269 and N-Hance 3270, which have a charge density of about 0.7 meq/g and a molecular weight of about 425,000 g/mol and are available from ASI; N-Hance 3196, which has a charge density of about 0.8 meq/g and a molecular weight of about 1,100,000 g/mol and is available from ASI. BF-13, which is a borate (boron) free guar of charge density of about 1.1 meq/g and molecular weight of about 800,000 and BF-17, which is a borate (boron) free guar of charge density of about 1.7 5 meq/g and molecular weight of about 800,000 both available from ASI. Another suitable guar hydroxypropyltrimonium chloride is Dehyquart Guar HP available from BASF.

The liquid compositions of the present disclosure may comprise a non-guar galactomannan polymer derivative, which has a mannose to galactose ratio of greater than about 2:1, or greater than about 3:1, or greater than about 4:1, on a monomer to monomer basis. Analysis of mannose to galactose ratios is well known in the art and is typically based on the measurement of the galactose content. The non-guar galactomannan polymer derivative may be a cationic galactomannan polymer derivative or an amphoteric galactomannan polymer derivative having a net positive charge. As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge. Non-guar galactomannan polymers are present in the endosperm of seeds of the Leguminosae family. Non-guar galactomannan polymers are made up of a combination of mannose monomers and galactose monomers. The galactomannan molecule is a straight chain mannan branched at regular intervals with single membered galactose units on specific mannose units. The mannose units are linked to each other by means of R (1-4) glycosidic linkages. The galactose branching arises by way of an a (1-6) linkage. The ratio of mannose monomers to galactose monomers varies according to the species of the plant and other factors, such as climate. The gum for use in preparing the non-guar galactomannan polymer derivatives is typically obtained as naturally occurring material such as seeds or beans from plants. Examples of various non-guar galactomannan polymers include but are not limited to Tara gum (3 parts mannose/1 part galactose), Locust bean or Carob (4 parts mannose/1 part galactose), and Cassia gum (5 parts mannose/1 part galactose). The non-guar galactomannan polymer derivatives may have a molecular weight from about 400,000 g/mol to about 10,000,000 g/mol, and/or from about 500,000 g/mol to about 5,000,000 g/mol. The personal care compositions of the invention can also include galactomannan polymer derivatives which have a cationic charge density from about 0.1 meq/g to about 3.0 meq/g. The non-guar galactomannan polymer derivatives may have a cationic charge density of about 0.6 meq/g to about 3 meq/g. The degree of substitution of the cationic groups onto the galactomannan structure should be sufficient to provide the preferred cationic charge density range described above.

The non-guar galactomannan polymer may be obtained by reaction between the hydroxyl groups of the polygalactomannan polymer and reactive quaternary ammonium compounds. Alternatively, the non-guar galactomannan polymer derivative can be an amphoteric derivative having a net positive charge, obtained when a cationic galactomannan polymer derivative further comprises an anionic group. Suitable cationic non-guar galactomannans may be derived from a cassia plant.

The liquid compositions of the present disclosure may also include ‘SoftCAT’ or ‘UCare’ polymers: The SoftCAT™ SL (SoftCAT™ SL, INCI name: Polyquaternium-67; Ballarin et al., 2011) constitute a family of high viscosity quaternized hydroxyethyl cellulose (HEC) polymers, with low cationic substitution, of trimethyl ammonium and dimethyldodecyl ammonium. “UCARE™ polymers (INCI Name: Polyquaternium-10) are polymeric, quaternary ammonium salts of hydroxyethylcellulose reacted with trimethyl ammonium substituted epoxide. The cellulosic backbone may be derived from natural, renewable resources.

### Ionic Surfactants

The liquid compositions of the present disclosure may be substantially free of ionic surfactants, such as anionic surfactants, cationic surfactants, amphoteric surfactants, and zwitterionic surfactants. Preferably, the liquid compositions are substantially free of anionic surfactants. Anionic surfactants include, but are not limited to, surface-active compounds that contain an organic hydrophobic group containing 8 to 22 carbon atoms or 8 to 18 carbon atoms and at least one water-solubilizing group, preferably selected from sulfonate, sulfate, and carboxylate, so as to form a water-soluble compound. Usually, the hydrophobic group will comprise a linear or branched C8-C22 alkyl, or acyl group. Such surfactants are employed in the form of water-soluble salts and the salt-forming cation usually is selected from sodium, potassium, ammonium, magnesium and mono-, di- or tri-alkanolammonium, with the sodium, cation being the usual one chosen. Without being bound by theory, it is believed that ionic surfactants, such as anionic surfactants, contribute to dispersing and redistributing soils, particularly oily or hydrophobic soils, by suspending the soils, for example, in surfactant micelles, and then redepositing the soils on the surface of the pet's hair/skin. For applications that involve rinsing with water, these suspended soils are readily washed away from the pet's hair/skin. However, in the context of an application involving a pre-lotioned wipe, where there is no rinse step, any residual lotion on the pet's hair/skin tends to redeposit soil and/or soil mixed with surfactant on the pet's hair/skin, as soil and/or soil mixed with surfactant (e.g., in a micelle) tends to be too small to be absorbed by the wipe substrate. Anionic surfactants may also react and interfere with the functioning of the cationic polymer. Anionic surfactants may also irritate a mammal's skin.

### Preservative

The liquid compositions of the present disclosure may comprise one or more preservatives. Preservatives may be synthetic or natural compounds, or a combination of compounds, that are effective in reducing or preventing the growth of microorganisms, thereby enabling a longer shelf life for a package of wet wipes (opened or not opened). The preservative may also create an environment that reduces or inhibits growth of microorganisms when the liquid composition is transferred to the skin during the wiping process. The preservative may be especially useful when a wet wipe liquid composition comprises a high concentration of water, for example, greater than about 96% water. The preservatives disclosed herein may be active against bacteria, molds, and/or yeast. The preservatives may reduce the growth rate of such microorganisms.

Natural preservatives may be derived from plants and/or animals, or from natural processes of plants and/or animals, such as fermentation. The liquid composition of the present disclosure may comprise a natural preservative, such as a 100% plant-based preservative. The preservative may consist of or consist essentially of natural compounds.

The preservative may comprise benzoic acid and/or a salt thereof. Benzoic acid is a carboxylic acid that occurs naturally in many plant and animal species. The liquid compositions of the present disclosure may comprise at least 0.1%, at least 0.14%, at least 0.17%, or from about 0.1% to about 0.6%, from about 0.1% to about 0.4%, from about 0.1% to about 0.3%, from about 0.1% to about 0.25%, from about 0.14% to about 0.4%, from about 0.14% to about 0.3%, from about 0.17% to about 0.3%, or from about 0.17% to about 0.25% of benzoic acid, by weight of the liquid composition, in the form of benzoic acid and/or a salt thereof, specifically reciting every 0.01% increment within these ranges and any ranges formed therein or thereby. The liquid composition may comprise sodium benzoate. The liquid composition may comprise benzoic acid and sodium benzoate.

The preservative may comprise succinic acid and/or a salt thereof. Succinic acid is a dicarboxylic acid that is found in many plant and animal sources, and may also be synthesized through fermentation by certain microorganisms. Succinic acid is used in the food industry as an acidity regulator and a flavoring agent. Succinic acid has unexpectedly been found to provide hostility against microorganisms in liquid compositions with a high water content. The liquid compositions of the present disclosure may comprise at least 0.15%, at least 0.2%, at least 0.25%, from about 0.15% to about 0.6%, or from about 0.1% to about 0.4%, from about 0.1% to about 0.3%, from about 0.2% to about 0.6%, from about 0.2% to about 0.45%, from about 0.2% to about 0.4%, from about 0.25% to about 0.4%, or from about 0.25% to about 0.35% of succinic acid, by weight of the liquid composition, in the form of succinic acid and/or a salt thereof, specifically reciting every 0.01% increment within these ranges and any ranges formed therein or thereby. The liquid composition may comprise sodium succinate. The liquid composition may comprise succinic acid and sodium succinate.

The preservative may comprise phytic acid and/or a salt thereof. Phytic acid is an organic acid that occurs naturally, for example, in many legumes, grains, and the bran of cereals. The liquid compositions of the present disclosure may comprise at least 0.02%, at least 0.04%, at least 0.06%, or from about 0.02% to about 0.2%, from about 0.02% to about 0.15%, from about 0.02% to about 0.1%, from about 0.04% to about 0.2%, from about 0.04% to about 0.15%, from about 0.04% to about 0.1%, from about 0.06% to about 0.1%, or from about 0.06% to about 0.08% of phytic acid, by weight of the liquid composition, in the form of phytic acid and/or a salt thereof, specifically reciting every 0.01% increment within these ranges and any ranges formed therein or thereby. The liquid composition may comprise sodium phytate. The liquid composition may comprise phytic acid and sodium phytate.

### Optional Adjunct Ingredients

The liquid composition may further comprise one or more optional, adjunct ingredients, including benefit agents, such as conditioning agents and/or natural oils (e.g., sunflower oil or castor oil). Additional suitable optional ingredients include but are not limited to chelating agents, emulsifiers, perfumes, perfume microcapsules, colorants, particles, anti-microbials, foam busters, anti-static agents, rheology modifiers and thickeners, suspension materials, structurants, pH adjusting agents, preservatives, pearlescent agents, sensates, anti-dandruff agents, solvents, diluents, anti-oxidants, vitamins, or combinations thereof.

Such adjunct ingredients are preferably physically and chemically compatible with the other components of the liquid composition. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting adjunct ingredients that can be added to the liquid compositions disclosed herein.

### Emulsifier

The liquid compositions may include one or more emulsifiers. Suitable emulsifiers include nonionic surfactants. The addition of an emulsifier may help solubilize or stabilize water insoluble or poorly water soluble actives or adjuncts such as perfume. The liquid compositions may comprise about 0.05 wt. % to less than 9.0 wt. %, or about 0.1 wt. % to about 8.0 wt. %, or about 0.1 wt. % to about 6.0 wt. %, of an emulsifier.

Suitable emulsifiers are selected from nonionic surfactants. Nonionic surfactants may comprise from about 9 to about 22 or from about 9 to about 18 or from about 9 to about 16 carbon atoms. Suitable nonionic surfactants include alkoxylated derivatives of sorbitan esters including, but not limited to, polyoxyethylene (20) sorbitan monolaurate (Tween® 20), polyoxyethylene (20) sorbitan monopalmitate (Tween® 40), polyoxyethylene (20) sorbitan monostearate (Tween® 60), polyoxyethylene (20) sorbitan monooleate (Tween® 80), polyoxyethylene (4) sorbitan monolaurate (Tween® 21), polyoxyethylene (4) sorbitan monostearate (Tween® 61), polyoxyethylene (5) sorbitan monooleate (Tween® 81), and mixtures thereof, all available from Uniqema.

Suitable nonionic surfactants also include those that can be broadly defined as condensation products of long chain alcohols, e.g., C8-30 alcohols, with sugar or starch polymers, i.e., glycosides. These compounds can be represented by the formula (S)ₙ—O—R where S is a sugar moiety such as glucose, fructose, mannose, and galactose; n is an integer of from about 1 to about 1000, and R is a C8-30 alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants include those where S is a glucose moiety, R is a C8-20 alkyl group, and n is an integer of from about 1 to about 9. Commercially available examples of these surfactants include decyl polyglucoside (available as APG 325 CS from Henkel) and lauryl polyglucoside (available as APG 600 CS and 625 CS from Henkel).

Suitable nonionic surfactants also include the condensation products of alkylene oxides with fatty acids (i.e., alkylene oxide esters of fatty acids). These materials have the general formula RCO(X)ₙOH where R is a C10-30 alkyl group, X is —OCH₂CH₂— (derived from ethylene glycol or ethylene oxide) or —OCH₂CHCH₃. (derived from propylene glycol or propylene oxide), and n is an integer from about 6 to about 200. Other suitable nonionic surfactants are the condensation products of alkylene oxides with 2 moles of fatty acids (i.e., alkylene oxide diesters of fatty acids). These materials have the general formula RCO(X)ₙOOCR, where R is a C10-30 alkyl group, X is —OCH₂CH₂— (derived from ethylene glycol or ethylene oxide) or —OCH₂CHCH₃— (derived from propylene glycol or propylene oxide), and n is an integer from about 6 to about 100. Other suitable nonionic surfactants include the condensation products of alkylene oxides with fatty alcohols (i.e., alkylene oxide ethers of fatty alcohols). These materials have the general formula R(X)ₙOR′, where R is a C10-30 alkyl group, X is —OCH₂CH₂— (derived from ethylene glycol or ethylene oxide) or —OCH₂CHCH₃. (derived from propylene glycol or propylene oxide), n is an integer from about 6 to about 100, and R′ is H or a C10-30 alkyl group. Still other suitable nonionic surfactants are the condensation products of alkylene oxides with both fatty acids and fatty alcohols, where the polyalkylene oxide portion is esterified on one end with a fatty acid and etherified (connected via an ether linkage) on the other end with a fatty alcohol. These materials have the general formula RCO(X)ₙOR′, where R and R′ are C10-30 alkyl groups, X is —OCH2CH2 (derived from ethylene glycol or ethylene oxide) or —OCH2CHCH3— (derived from propylene glycol or propylene oxide), and n is an integer from about 6 to about 100. Nonlimiting examples of these alkylene oxide derived nonionic surfactants include ceteth-6, ceteth-10, ceteth-12, ceteareth-6, ceteareth-10, ceteareth-12, steareth-6, steareth-10, steareth-12, steareth-21, PEG-6 stearate, PEG-10 stearate, PEG-100 stearate, PEG-12 stearate, PEG-20 glyceryl stearate, PEG-80 glyceryl tallowate, PEG-10 glyceryl stearate, PEG-30 glyceryl cocoate, PEG-80 glyceryl cocoate, PEG-200 glyceryl tallowate, PEG-8 dilaurate, PEG-10 distearate, and mixtures thereof.

Additional suitable nonionic surfactants include polyhydroxy fatty acid amide surfactants, sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, and mixtures thereof. Nonlimiting examples of these emulsifiers include: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, PPG-2 methyl glucose ether distearate, PEG-100 stearate, and mixtures thereof. Another group of suitable non-ionic surfactants are fatty acid ester blends based on a mixture of sorbitan or sorbitol fatty acid ester and sucrose fatty acid ester, the fatty acid in each instance being preferably C8-C24, more preferably C10-C20.

### Humectant

The liquid compositions may include one or more humectants. The addition of a humectant may decrease the water activity of the liquid composition and/or reduce the weight loss rate of the liquid composition over time due to water evaporation. The liquid compositions my comprise about 0.05 wt. % to about 3.0 wt % of one or more humectants.

### Triethyl Citrate

The liquid composition of the present disclosure may comprise triethyl citrate. The liquid composition may comprise from about 0.01 wt. % to 5.0 wt. %, or from about 0.05 wt. % to 2 wt. %, or from about 0.1 wt. % to 1.0 wt. % triethyl citrate. Without wishing to be bound by theory, the triethyl citrate may promote phase stability in the liquid composition, particularly a liquid composition that contains perfume.

### Chelating Agent

The liquid compositions of the present disclosure may comprise a chelant. Suitable chelants include those listed in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference. When related to chelants, the term “salts and derivatives thereof” means the salts and derivatives comprising the same functional structure (e.g., same chemical backbone) as the chelant they are referring to and that have similar or better chelating properties. Chelating agents can be incorporated in the compositions herein in amounts ranging from about 0.001% to about 10.0%, or about 0.01% to 2.0% by weight of the composition. Chelating agents may be divided into several classes including carboxylic acids, aminocarboxylic acids, phosphoric acids, phosphonic acids, polyphosphonic acids, polyethyleneimines, polyfunctionally-substituted aromatics, derivatives or salts thereof, and mixtures thereof.

Suitable chelating agents include the following materials and their salts: ethylenediaminetetraacetic acid (EDTA), ethylenediaminetriacetic acid, ethylenediamine-N,N′-disuccinic acid (EDDS), ethylenediamine-N,N′-diglutaric acid (EDDG), salicylic] acid, aspartic acid, glutamic acid, glycine, malonic acid, histidine, diethylenetriaminepentaacetate (DTPA), N-hydroxyethylethylenediaminetriacetate, nitrilotriacetate, ethylenediaminetetrapropionate, triethylenetetraaminehexaacetate, ethanoldiglycine, propylenediaminetetracetic acid (PDTA), methylglycinediacetic acid (MODA), diethylenetriaminepentaacetic acid, methylglycinediacetic acid (MGDA), N-acyl-N,N′,N′-ethylenediaminetriacetic acid, nitrilotriacetic acid, ethylenediaminediglutaric acid (EDGA), 2-hydroxypropylenediamine disuccinic acid (HPDS), glycinamide-N, N′-disuccinic acid (GADS), 2-hydroxypropylenediamine-N-N′-disuccinic acid (HPDDS), N-2-hydroxyethyl-N,N-diacetic acid, glyceryliminodiacetic acid, iminodiacetic acid-N-2-hydroxypropyl sulfonic acid, aspartic acid N-carboxymethyl-N-2-hydroxypropyl-3-sulfonic acid, alanine-N,N′-diacetic acid, aspartic acid-N,N′-diacetic acid, aspartic acid N-monoacetic acid, iminodisuccinic acid, diamine-N,N′-dipolyacid, monoamide-N,N′-dipolyacid, diaminoalkyldi(sulfosuccinic acids) (DDS), ethylenediamine-N-N′-bis (ortho-hydroxyphenyl acetic acid)), N,N′-bis(2-hydroxybenzyl)ethylenediamine-N, N′-diacetic acid, ethylenediaminetetraproprionate, triethylenetetraaminehexacetate, diethylenetriaminepentaacetate, dipicolinic acid, ethylenedicysteic acid (EDC), ethylenediamine-N,N′-bis(2-hydroxyphenylacetic acid) (EDDHA), glutamic acid diacetic acid (GLDA), hexadentateaminocarboxylate (HBED), polyethyleneimine, 1-hydroxydiphosphonate, aminotri(methylenephosphonic acid) (ATMP), nitrilotrimethylenephosphonate (NTP), ethylenediaminetetramethylenephosphonate, diethylenetriaminepentamethylenephosphonate (DTPMP), ethane-1-hydroxydiphosphonate (HEDP), 2-phosphonobutane-1,2,4-tricarboxylic acid, polvphosphoric acid, sodium tripolyphosphate, tetrasodium diphosphate, hexametaphosphoric acid, sodium metaphosphate, phosphonic acid and derivatives, Aminoalkylen-poly(alkylenphosphonic acid), aminotri(1-ethylphosphonic acid), ethylenediaminetetra(1-ethylphosphonic acid), aminotri(1-propylphosphonic acid), aminotri(isopropylphosphonic acid), ethylenediaminetetra(methylenephosphonic acid) (EDTMP), 1,2-dihydroxy-3,5-disulfobenzene, and mixtures thereof.

### Emollients

The liquid compositions of the present disclosure may comprise an emollient or emollients. Emollients may (1) hydrate soil residues, thus enhancing their removal from the hair and/or skin, (2) hydrate the skin, thus reducing its dryness and irritation, (3) protect the skin from later irritation, as the emollient is deposited onto the skin and may remain there as a thin protective layer, and/or (4) provide a desired sensory feel to the liquid composition and/or the skin.

### Rheology Modifiers

The liquid compositions of the present disclosure may comprise one or more rheology modifiers. Non-limiting examples of rheology modifiers include, but are not limited to, hydrocolloids, including natural gums, such as xanthan gum. The liquid compositions of the present disclosure may comprise from about 0.01% to about 0.1%, from about 0.03% to about 0.08%, from about 0.05% to about 0.07%, or about 0.06% of a rheology modifier, by weight of the liquid composition, specifically reciting every 0.01% increment within these ranges and every range formed therein or thereby.

### Perfume

The liquid compositions of the present disclosure may comprise a perfume or fragrance. The liquid composition may comprise from about 0.025 wt % to about 2 wt %, preferably about 0.1 wt % to about 0.5 wt %, of a perfume or fragrance. The perfume or fragrance may be a natural compound or compounds. The perfume may be designed and/or selected to appeal to both pet owners and pets. For example, pet-friendly perfumes, such as those available under the NeoFresh® tradename from Symrise, may help to suppress odors on a pet without the pet perceiving the smell of the perfume as unpleasant. US Patent Publication No. 2021/0275082 describes a method for measuring pet acceptance of perfumes and identifying pet preferred perfumes. Alternatively, the wet wipe of the present disclosure may be substantially free of perfume and/or fragrance.

### Wet Wipe Substrates

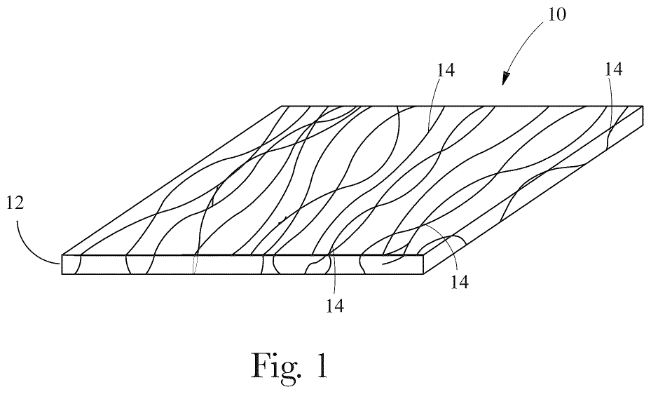

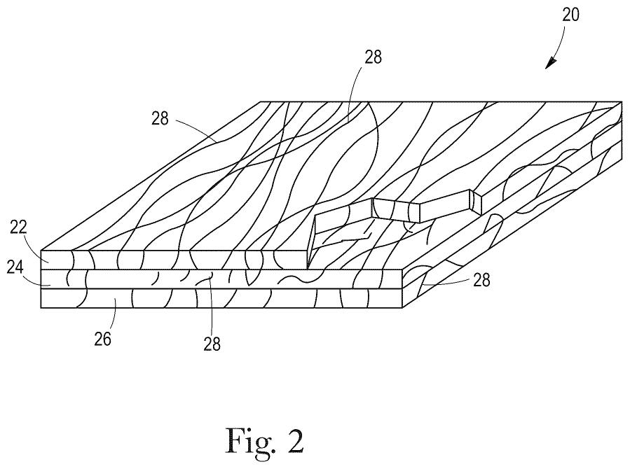

Wet wipes are typically constructed from porous or absorbent sheets of substrate saturated with a liquid composition and are sold and stored in an air-tight container or wrapper to prevent, or at least inhibit, the sheets from drying out. The liquid compositions of the present disclosure may be loaded onto a substrate, such as a nonwoven substrate, to form a wet wipe. The substrate may be homogenous or may be layered. If layered, the substrate may comprise at least two, at least three, at least four, or at least five layers. Referring to FIG. 1, the substrate 10 may be formed of one layer 12 of a web of fibers 14. Referring to FIG. 2, the substrate 20 may be formed of more than one layer of webs of fibers 28. For example, the substrate 20 may be formed of a first layer 22 of a web of fibers 28, a second layer 24 of a web of fibers 28, and a third layer 26 of a web of fibers 28. The one or more layers of webs of fibers may comprise a nonwoven web of materials comprising continuous fibers, coextruded fibers, non-continuous fibers, and/or combinations thereof.

The substrate (e.g., nonwoven substrate) may comprise natural fibers, e.g., cellulosic fibers, synthetic fibers, or a combination thereof. Natural fibers include cellulosic fibers, such as fibers from hardwood sources, softwood sources, or other non-wood plants. Non-limiting examples of cellulosic fibers include, but are not limited to, wood pulp, typical northern softwood Kraft, typical southern softwood Kraft, typical CTMP, typical deinked, corn pulp, acacia, eucalyptus, aspen, reed pulp, birch, maple, radiata pine, albardine, esparto, wheat, rice, corn, sugar cane, papyrus, jute, reed, sabia, raphia, bamboo, sidal, kenaf, abaca, cotton, flax, hemp, jute, modified natural cellulosic fibers such as, for example, rayon (including viscose, lyocell, MODAL (a product of Lenzing AG, Lenzing, Austria) and cuprammonium rayon), and combinations thereof. Cellulosic fibers may be consumer-preferred to appeal to a desire for natural and/or environmentally friendly products. The natural fibers may be treated or otherwise modified mechanically or chemically to provide desired characteristics or may be in a form that is generally similar to the form in which they can be found in nature. Mechanical and/or chemical manipulation of natural fibers does not exclude them from what are considered natural fibers with respect to the present disclosure. For example, cellulosic fibers may be treated or otherwise modified mechanically and/or chemically to provide desired characteristics, or may be in a form that is generally similar to the form in which they may be found in nature. Thus, fibers derived from cellulose, such as regenerated cellulose (e.g., viscose), are also considered as cellulose fibers according to the present disclosure. The substrate may comprise cellulosic fibers, modified cellulosic fibers, synthetic fibers, mixtures of cellulosic and/or modified cellulosic fibers with synthetic fibers, and/or combinations thereof.

Non-limiting examples of synthetic fibers include polyesters (e.g., polyethylene terephthalate), polyolefins, polypropylenes, polyethylenes, polyethers, polyamides, polyesteramides, polyvinylalcohols, polyhydroxyalkanoates, polysaccharides, and combinations thereof. The polyester may comprise less than 100 ppm antimony, or the polyester may be devoid of antimony. Further, the synthetic fibers may be a single component (i.e., single synthetic material or mixture makes up entire fiber), bi-component (i.e., the fiber is divided into regions, the regions including two or more different synthetic materials or mixtures thereof and may include co-extruded fibers and core and sheath fibers) and combinations thereof. Bi-component fibers may be used as a component fiber of the structure, and/or they may be present to act as a binder for the other fibers present in the fibrous structure. Any or all of the synthetic fibers may be treated before, during, or after manufacture to change any desired properties of the fibers. The fibers of the substrate may be processed to be suitably soft-feeling against the skin. The substrate may comprise hydrophilic fibers, hydrophobic fibers, or a combination thereof.

The wet wipe substrate may comprise about 0% to about 90%, or about 1% to about 90%, or about 1% to about 85%, or about 5% to about 55%, or about 10% to about 35%, by weight of the substrate of synthetic fibers. The substrate may comprise about 10% to about 100%, or about 10% to about 65%, or about 14% to about 45%, by weight of the substrate of natural fibers. The substrate may comprise between about 30% and about 100%, or between about 50% and about 100%, or between about 65% and 100%, by weight of the substrate of natural, preferably cellulosic, fibers, specifically reciting each 1% increment within these ranges and every range formed therein or thereby. The substrate may comprise about 50% natural, preferably cellulosic, fibers and about 50% synthetic fibers, by weight of the substrate. The substrate may comprise about 30% natural, preferably cellulosic, fibers and about 70% synthetic fibers, by weight of the substrate. The substrate may comprise about 100% natural, preferably cellulosic, fibers, such as a mixture of about 50% viscose and about 50% lyocell or a mixture of 85% lyocell and about 15% cotton, by weight of the substrate. The substrate may comprise about 100% natural, preferably cellulosic, fibers, where the cellulosic fibers comprise between about 10% to about 70% viscose, by weight of the substrate. The substrate may be substantially free of polyamides.

It may be desirable that the substrate, or at least one or more layers of the substrate, comprises a particular combinations of fibers to provide desired characteristics. For example, it may be desirable to have fibers of certain lengths, widths, coarseness, or other characteristics combined in certain layers, or separate from each other. The fibers may be of virtually any size and may have an average length from about 1 mm to about 60 mm, specifically reciting each 1 mm increment within the range and every range formed therein. Average fiber length refers to the length of the individual fibers if straightened out. The fibers may have an average fiber width of greater than about 5 micrometers. The fibers may have an average fiber width of from about 5 micrometers to about 50 micrometers, specifically reciting each 1 micrometer increment within the range and every range formed therein. The fibers may have a coarseness of greater than about 5 mg/100 m. The fibers may have a coarseness of from about 5 mg/100 m to about 75 mg/100 m, specifically reciting each 1 mg/100 m increment within the range and every range formed therein.

The fibers may be circular in cross-section, dog-bone shape, delta (i.e., triangular cross section), trilobal, ribbon, or other shapes typically produced as staple fibers. Likewise, the fibers may be conjugate fibers such as bicomponent fibers. The fibers may be crimped and may have a finish, such as a lubricant, applied.

The materials comprising the substrate may be treated to improve the softness and texture thereof. The substrate may be subjected to various treatments, such as physical treatment, hydro-molding, hydro-embossing, hydro-entangling, ring rolling, as described in U.S. Pat. No. 5,143,679; structural elongation, as described in U.S. Pat. No. 5,518,801; consolidation, as described in U.S. Pat. Nos. 5,914,084; 6,114,263; 6,129,801 and 6,383,431; stretch aperturing, as described in U.S. Pat. Nos. 5,628,097; 5,658,639; and 5,916,661; differential elongation, as described in U.S. Pat. No. 7,037,569, other solid state formation technologies as described in U.S. Pat. Nos. 7,553,532 and 7,410,683; zone activation, and the like; chemical treatment, such as rendering part or all of the substrate hydrophobic and/or hydrophilic, and the like; thermal treatment, such as thermal-embossing, softening of fibers by heating, thermal bonding and the like; and combinations thereof.

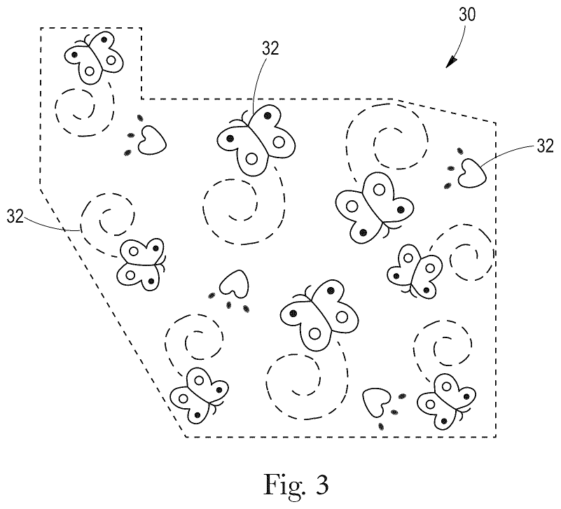

Without wishing to be bound by theory, it is believed that a textured substrate may further enable the ease of removal of soils by improving the ability to grip or otherwise lift the soils from the surface during cleansing. Any one of a number of texture elements may be useful in improving the ability to grip or otherwise lift the soil from the surface during cleansing, such as continuous hydro-molded elements, hollow molded element, solid molded elements, circles, squares, rectangles, ovals, ellipses, irregular circles, swirls, curly cues, cross hatches, pebbles, lined circles, linked irregular circles, half circles, wavy lines, bubble lines, puzzles, leaves, outlined leaves, plates, connected circles, changing curves, dots, honeycombs, and the like, and combinations thereof. The texture elements may be hollow elements. The texture elements may be connected to each other. The texture elements may overlap each other. The texture elements may form a pattern. Referring to FIG. 3, the substrate 30 may comprise a plurality of texture elements 32 that form a pattern in and/or on the substrate 30.

The substrate may have a basis weight from about 15 g/m² (gsm) to about 100 g/m² (gsm), or from about 40 g/m² (gsm) to about 100 g/m² (gsm), or from about 40 g/m² (gsm) to about 85 g/m² (gsm), or from about 45 g/m² (gsm) to about 80 g/m² (gsm), specifically reciting every 1 g/m² (gsm) increment within the ranges and every range formed therein or thereby. Exemplary nonwoven substrates are described in U.S. Patent Publication 2012/066852 and U.S. Patent Publication U.S. 2011/244199.

The surface of the substrate may be essentially flat. The surface of the substrate may optionally contain raised and/or lowered portions. The raised and/or lowered portions may be in the form of logos, indicia, trademarks, geometric patterns, and/or images of the surfaces that the substrate is intended to clean (e.g., dog hair/fur, including hair/fur on paws). The raised and/or lowered portions may be randomly arranged on the surface of the substrate or be in a repetitive pattern.

The substrate may be biodegradable. For example, the substrate may comprise a biodegradable material such as a polyesteramide or a high wet strength cellulose. The substrate may be dispersible in water.

The substrates described herein may have different properties on different sides of the substrate. For example, one side of the substrate may have good cleaning performance and the other side of the substrate may have good tactile sensation to the user. In another form, one side of the substrate may have an increased cleaning performance as compared to the other side of the substrate.

Non-limiting examples of processes for making webs of fibers of the substrate described herein include known wet-laid papermaking processes, air-laid papermaking processes including carded and/or spunlaced processes. Such processes typically include steps of preparing a fiber composition in the form of a suspension in a medium, either wet, more specifically aqueous medium, or dry, more specifically gaseous, i.e., with air as a medium. The aqueous medium used for wet-laid processes is oftentimes referred to as a fiber slurry. The fibrous slurry is then used to deposit a plurality of fibers onto a forming wire or belt such that an embryonic web of fibers is formed, after which drying and/or bonding the fibers together results in a web of fibers. Further processing the web of fibers may be carried out such that a finished web of fibers is formed. For example, in typical papermaking processes, the finished web of fibers is the fibrous structure that is wound on the reel at the end of papermaking, and may subsequently be converted into a finished product, e.g., a wet wipe substrate.

The web of fibers of the substrates described herein may be a co-formed fibrous structure. “Co-formed fibrous structure,” as used herein, means that the fibrous structure comprises a mixture of at least two different materials, where at least one of the materials comprises a filament, such as a polypropylene filament, and at least one other material different from the first material, comprises a solid additive, such as a fiber and/or a particulate. In one example, a co-formed fibrous structure comprises solid additives—such as fibers (for example, wood pulp fibers) and/or absorbent gel materials and/or filler particles and/or particulate spot bonding powders and/or clays—and filaments—such as polypropylene filaments. “Solid additive” as used herein means a fiber and/or a particulate. “Particulate” as used herein means a granular substance or powder. “Fiber” and/or “Filament” as used herein means an elongate particulate having an apparent length greatly exceeding its apparent width, i.e., a length to diameter ratio of at least about 10. For purposes of the present disclosure, a “fiber” is an elongate particulate as described above that exhibits a length of less than 5.08 cm (2 in.) and a “filament” is an elongate particulate as described above that exhibits a length of greater than or equal to 5.08 cm (2 in.).

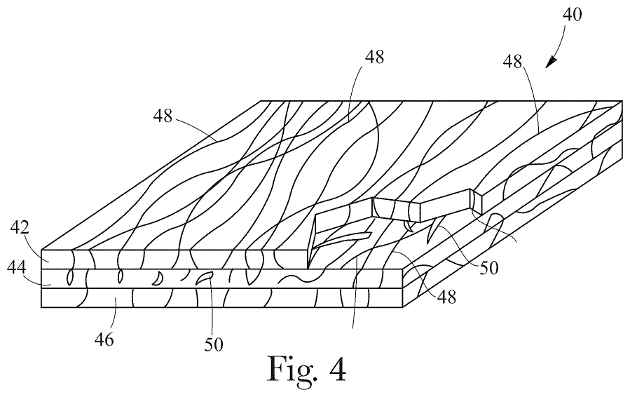

Referring to FIG. 4, the substrate 40 may be a co-formed layered fibrous structure. The co-formed layered fibrous structure may comprise a first layer 44 comprising a plurality of filaments 48, such as polypropylene filaments, and a plurality of solid additives 50, such as wood pulp fibers, for example. The co-formed layered fibrous structure 40 may further comprise a second layer 42 and a third layer 46, each comprising a plurality of filaments 48, such as polypropylene filaments, for example. The first layer 44 may be disposed between the second layer 42 and the third layer 46. In a form, the first, second, and third layers may be sharply defined zones of concentration of the filaments and/or solid additives. The plurality of filaments of the second and third layers may be deposited directly onto a surface of the first layer to form the co-formed layered fibrous structure. In another form, the first layer may form an outward-facing surface of the substrate and may be in direct contact with only one other layer.

The substrate described herein may be subjected to any post-processing operations such as embossing operations, printing operations, tuft-generating operations, thermal bonding operations, ultrasonic bonding operations, perforating operations, surface treatment operations such as application of liquid compositions or lotions, silicones and/or other materials, folding, and/or combinations thereof.

Without being bound by theory, it is believed that different compounds present in a liquid composition may interact to different degrees with the various kinds of nonwoven fibers that are present in the substrate that the liquid composition is loaded on to make the wet wipe. Different kinds of interactions may occur based on the physical and chemical properties of the liquid composition compound and of the nonwoven fibers. These interactions may lead to adsorption of the liquid composition compound to the surface of the fiber or to absorption of the liquid composition compound into the structure of the fiber.

Hansen's solubility parameters are a way of predicting if one material may tend to interact with, and potentially absorb into or adsorb to, another material. Each molecule is given three Hansen parameters, one for the dispersion forces between molecules, one for the dipolar intermolecular force between molecules, and one for the hydrogen bonding between molecules. These parameters serve as coordinates for a point in three dimensions known as the Hansen space. The nearer two molecules are in this three-dimensional space, the more compatible they are, the more likely they are to interact, and the greater the potential for them to absorb into or adsorb to each other.

For example, in the case of polyester fibers, certain organic acids, such as benzoic acid, share physical and chemical similarities to polyester that may cause them to have a fairly similar Hansen space. This means that, over time, while a liquid composition is in contact with a wipe substrate comprising polyester fibers, certain organic acids, such as benzoic acid, and/or a salt thereof, may interact with (absorb into or adsorb onto) the polyester fibers, reducing their concentration in the liquid composition and making them unavailable to participate in preservative efficacy.

### Loading Ratio

The liquid compositions of the present disclosure may be incorporated onto a substrate at a load of about 200% to about 600%, from about 300% to about 500%, or from about 325% to about 460%, by weight of the substrate, specifically reciting every 1% increment within these ranges and every range formed therein or thereby. Said differently, the liquid compositions of the present disclosure may be incorporated onto a substrate in a loading ratio of liquid composition to substrate of about 2.0 g/g to about 6.0 g/g, or about 2.0 g/g to about 5.0 g/g/, or about 3.0 g/g to about 4.5 g/g.

### EXAMPLES

The following examples and comparative examples are provided to help illustrate the liquid compositions and substrates described herein. The exemplified liquid compositions may be prepared by conventional formulation and mixing techniques. It will be appreciated that other modifications of the liquid compositions described herein within the skill of those in the formulation art may be undertaken. All parts, percentages, and ratios herein are by weight unless otherwise specified.

Table 1 shows the formulations of the liquid compositions of Examples A-F. Table 2 describes examples and comparative examples comprising varying substrates, liquid compositions, and loading ratios. In Table 2, substrate S1 is a hydromolded spunlace substrate comprising an 80:20 blend of polyethylene terephthalate (PET) and viscose, while substrate S2 is a spunbond substrate comprising 50% polypropylene (PP) and 50% polyethylene (PE).

Examples 1 and 2 and Comparative Examples 1-3 have varying substrate compositions, substrate basis weights, and/or substrate absorption capacities, but contain the same liquid composition. Comparative Examples 4, 5, and 6 contain different liquid compositions, but have the same substrates. The liquid compositions of Comparative Examples 4, 5, and 6 respectively contain 10% emulsifier (nonionic surfactant), 0.3% anionic surfactant (sodium lauryl sulfate), and 0.3% amphoteric surfactant (cocamidopropyl betaine). Comparative Example 4 is observed to have an unpleasant odor. Comparative Examples 5 and 6 contain ionic surfactants, which may interfere with the cationic polymer and have other drawbacks as well, as discussed above.

TABLE 1

| | A | B | C | D | E | F |
|---|---|---|---|---|---|---|
| Cationic | 0.06 | 0.06 | 0.06 | 0.06 | 0.06 | 0.06 |
| Polymer¹ | | | | | | |
| Hexanediol² | 0.1-0.2 | 0.1-0.2 | 0.1-0.2 | 0.1-0.2 | 3.5-3.6 | 0.1-0.2 |
| Caprylyl | 0.1-0.2 | 0.1-0.2 | 0.1-0.2 | 0.1-0.2 | 0.1-0.2 | 0.1-0.2 |
| Glycol² | | | | | | |
| Chelating | 0.1 | 0.1 | 0.1 | 0.1 | 0.1 | 0.1 |
| Agent³ | | | | | | |
| Emulsifier | 0.3⁴ | 10⁴ | 0 | 0 | 0.45⁵ | 2.8⁶ |
| Ionic | 0 | 0 | 0.3⁷ | 0.3⁸ | 0 | 0 |
| Surfactant | | | | | | |
| Triethyl Citrate | 0 | 0 | 0 | 0 | 0.25 | 0 |
| Preservative⁹ | 0.2 | 0.2 | 0.2 | 0.2 | 0.2 | 0.2 |
| Perfume | 0.1 | 0.1 | 0.1 | 0.1 | 0.1 | 0.1 |
| pH Adjusting | 0.1 | 0.1 | 0.1 | 0.1 | 0.1 | 0.1 |
| agent¹⁰ | | | | | | |
| pH | 4.5 | 4.5 | 4.5 | 4.5 | 4.5 | 4.5 |

¹Styleze ™ CC-10 (2-propenamide, N-(3-dimethylamino)propyl-2-methyl-, polymer with 1-ethenyl-2-pyrrolidinone, sulfate), available from Ashland.
²Symdiol ® 68 (blend of 1,2-hexanediol and 1,2-octanediol), available from Symrise.
³Disodium EDTA.
⁴Tween ™ 80 (ethoxylated (20) sorbitan monooleate), available from Croda.
⁵Decyl glucoside.
⁶TEGO ™ Solve 90 MB (polyglyceryl-6 caprylate, polyglyceryl-4 caprate), available from Evonik.
⁷Sodium lauryl sulfate.
⁸Cocamidopropyl betaine.
⁹Sodium benzoate.
¹⁰Citric acid.

TABLE 2
Examples 1-4 (E1-E4) and Comparative Examples 1-6 (CE1-CE6).

| | E1 | E2 | E3 | E4 | CE1 | CE2 | CE3 | CE4 | CE5 | CE6 | CE7 | CE8 |
|---|---|---|---|---|---|---|---|---|---|---|---|---|
| Substrate | S1 | S1 | S1 | S1 | S1 | S1 | S2 | S1 | S1 | S1 | S1 | S1 |
| Composition | | | | | | | | | | | | |
| Substrate basis | 48¹¹ | 80¹² | 80¹² | 80¹² | 35¹¹ | 40¹² | 80 | 80¹² | 80¹² | 80¹² | 80¹² | 80¹² |
| weight (g/m²) | | | | | | | | | | | | |
| Substrate | 10 | 9.9 | 9.9 | 9.9 | 12 | 15.7 | 0 | 9.9 | 9.9 | 9.9 | 9.9 | 9.9 |
| Absorption | | | | | | | | | | | | |
| Capacity¹³ | | | | | | | | | | | | |
| (g/g) | | | | | | | | | | | | |
| Liquid | A | A | E | F | A | A | A | B | C | D | A | N/A |
| composition | | | | | | | | | | | | |
| from Table 1 | | | | | | | | | | | | |
| Loading Ratio | 4.5 | 4.5 | 3.5 | 3 | 4.5 | 4.5 | 4.5 | 4.5 | 4.5 | 4.5 | 0.5 | 0.0 |
| (g/g) | | | | | | | | | | | | |

¹¹Substrate available from Spuntech Industries, Inc., Roxboro, NC.
¹²Substrate available from Suominen of Tampere, Finland.
¹³Substrate Absorption Capacity as reported by the substrate manufacturer.

Results from technical testing of Example 1, Example 2, Comparative Example 1, Comparative Example 2, and Comparative Example 3 are shown in Table 3. The wet wipes of Examples 1 and 2, having respective basis weights of 48 g/m² and 80 g/m² and respective absorption capacities of 10 g/g and 9.9 g/g, “pass” the Black Glove Test. Based on the results of the Black Glove test, the wipes of Examples 1 and 2 are expected to keep a user's hands clean and free of soil, while he or she uses the wipes to clean a pet, such as a dog. The wipes of Examples 1 and 2 also have respective scores of 69 and 56 on the Tile Test, which are not significantly different from the clean black tile, meaning that the wipes of Examples 1 and 2 effectively clean the tile by removing the soil rather than redispersing it. Based on the results of the Tile Test, the wipes of Examples 1 and 2 are expected to be effective for removing soil from a pet, such as a dog, without redistributing the soil.

The wet wipes of Comparative Examples 1 and 2, having respective basis weights of 35 g/m² and 40 g/m² and respective absorption capacities of 12 g/g and 15.7 g/g, have scores of 64 and 75 on the Tile Test, which are not significantly different from the clean black tile, but each “fail” the Black Glove Test. Based on the results of the Black Glove test, the wipes of Comparative Examples 1 and 2 are expected to transfer removed soil to a user's hand. The wet wipe of Comparative Example 3, which has a basis weight of 80 g/m² and an absorption capacity of 0 g/g, has a score of 74 on the Tile Test, which is not significantly different from the clean black tile, but also “fails” the Black Glove Test. Based on the results of the Black Glove test, the wipe of Comparative Example 3 is expected to transfer removed soil to a user's hand.

TABLE 3A

| | Control | | | | | |
|---|---|---|---|---|---|---|
| | (clean tile) | E1 | E2 | CE1 | CE2 | CE3 |
| Tile Test | 64 | 67 | 59 | 64 | 75 | 74 |
| Black Glove Test | N/A | Pass | Pass | Fail | Fail | Fail |

As shown in Table 4, the loading ratio of liquid composition to substrate also affects cleaning performance, as evaluated by the Tile Test. Comparative examples 7 and 8 and Example 2 comprise the same substrate, loaded with 0.5 g/g of Composition A, 0 g/g of Composition A, and 4.5 g/g of Composition A, respectively.

TABLE 4

| | Control (clean tile) | E2 | CE7 | CE8 |
|---|---|---|---|---|
| Tile Test | 64 | 59 | 153 | 161 |

Comparative Examples 9-11: The wet wipes described as Comparative Examples 9-11 are commercially purchased wet wipe products containing the ingredients listed below, as disclosed on the ingredients listing of each product.

Comparative Example 9: The wet wipe described as Comparative Example 9 is a PAMPERS® Sensitive comprising water, citric acid, PEG-40 hydrogenated castor oil, sodium citrate, sorbitan caprylate, sodium benzoate, disodium EDTA, isoamyl laurate, and xanthan gum.

Comparative Example 10: The wet wipe described as Comparative Example 10 is a POGI'S® Plant-Based Dog Grooming Wipes comprising purified water, aloe vera extract, vitamin E, awapuhi extract, cucumber extract, Polysorbate 20, and benzoic acid.

Comparative Example 11: The wet wipe described as Comparative Example 11 is a TROPICLEAN® Berry & Coconut Deep Cleaning Pet Wipes comprising purified water, mild coconut cleanser, chamomile extract, aloe extract, Ginkgo biloba leaf extract, lavender flower extract, green tea extract, tea tree extract, citric acid, witch hazel extract, dipropylene glycol, glycerine, fragrance, and preservative.

The liquid compositions of the wet wipes described as Comparative Examples 9-11 have surface tensions that are similar to the surface tension of the liquid composition of the wet wipe of Example 2. However, the cleaning performances of the wipes of Comparatives Examples 9-11 are inferior to the cleaning performance of the wipe of Example 2. As shown in Table 5, the wet wipes of Comparative Examples 9, 10, and 11 have respective scores of 147, 112, and 118 on the Tile Test, which are significantly different than the clean black tile, meaning these wipes have inferior cleaning performance. In contrast, the wet wipe of Example 2 has a score of 59 on the Tile Test, which is not significantly different from the color of the clean black tile, indicating good cleaning performance.

TABLE 5

| SFT | E2 | CE9 | CE10 | CE11 |
|---|---|---|---|---|
| (mN/m) | 36 | 35 | 36 | 35 |
| Tile Test | 59 | 147 | 120 | 118 |

### Combinations:

A. A wet wipe comprising: (a) a liquid composition comprising: (i) about 0.01% to about 0.2% by weight of the composition of a cationic polymer, where the cationic polymer has a calculated cationic charge density of about 0.1 to about 4.0, preferably about 0.4 to about 3.2, more preferably about 0.75 to about 2.5, and a weight average molecular weight of about 10,000 g/mol to about 1,750,000 g/mol, preferably about 10,000 g/mol to about 1,600,000 g/mol, more preferably about 10,000 g/mol to about 1,500,000 g/mol; (ii) about 0.05 to about 4.0% by weight of the composition of a cleaning solvent selected from the group consisting of hexanediol, caprylyl glycol, and mixtures thereof; (iii) about 95% to about 99.5% by weight of the composition of water; where the composition has a pH of about 3 to about 6; and (b) a non-woven substrate comprising: (i) about 0% to about 90%, preferably about 1% to about 85%, more preferably about 5% to about 55%, even more preferably about 10% to about 35%, by weight of the substrate of synthetic fibers; (ii) about 10% to about 100%, preferably about 10% to about 65%, more preferably about 14% to about 45%, by weight of the substrate of natural fibers; where the non-woven substrate has a basis weight from about 40 g/m² to about 100 g/m², preferably about 40 g/m² to about 85 g/m², more preferably about 45 g/m² to about 80 g/m²; where the loading ratio of liquid composition to non-woven substrate is about 2.0 g/g to about 6.0 g/g, preferably about 2.5 g/g to about 5.0 g/g, more preferably about 3.0 g/g to about 4.5 g/g.
B. A wet wipe comprising: (a) a liquid composition comprising: (i) about 0.01% to about 0.2% by weight of the composition of a cationic polymer, where the cationic polymer has a calculated cationic charge density of about 0.1 to about 4.0, preferably about 0.4 to about 3.2, more preferably about 0.75 to about 2.5, and a weight average molecular weight of about 10,000 g/mol to about 1,750,000 g/mol, preferably about 10,000 g/mol to about 1,600,000 g/mol, more preferably about 10,000 g/mol to about 1,500,000 g/mol; (ii) about 80% to about 99.5% by weight of the composition of water; where the composition has a surface tension of about 30 mN/m to about 60 mN/m and a pH of about 3 to about 6; and (b) a non-woven substrate comprising: (i) about 0% to about 90%, preferably about 1% to about 85%, more preferably about 5% to about 55%, even more preferably about 10% to about 35%, by weight of the substrate of synthetic fibers; (ii) about 10% to about 100%, preferably about 10% to about 65%, more preferably about 14% to about 45%, by weight of the substrate of natural fibers; where the non-woven substrate has a basis weight from about 40 g/m² to about 100 g/m², preferably about 40 g/m² to about 85 g/m², more preferably about 45 g/m² to about 80 g/m²; where the loading ratio of liquid composition to non-woven substrate is about 2.0 g/g to about 6.0 g/g, preferably about 2.5 g/g to about 5.0 g/g, more preferably about 3.0 g/g to about 4.5 g/g.
C. The wet wipe according to any one of the preceding paragraphs, where the non-woven substrate has an absorption capacity of about 9 g/g to about 11 g/g.
D. The wet wipe according to any one of the preceding paragraphs, where the substrate is substantially free of polyamides.
E. The wet wipe according to any one of the preceding paragraphs, where the natural fibers comprise cellulose fibers, preferably viscose fibers.
F. The wet wipe according to any one of the preceding paragraphs, where the liquid composition is substantially free of ionic surfactants, preferably substantially free of anionic surfactants.
G. The wet wipe according to any one of the preceding paragraphs, where the liquid composition comprises a pH adjuster, preferably citric acid.
H. The wet wipe according to any one of the preceding paragraphs, where the area of the wet wipe is about 100 cm² to about 1000 cm², preferably about 100 cm² to about 800 cm², more preferably about 200 cm² to about 700 cm², even more preferably about 200 cm² to about 500 cm².
I. The wet wipe according to any one of the preceding paragraphs, where the wet wipe is disposable.
J. The wet wipe according to any one of the preceding claims, where the caliper of the wet wipe is greater than about 0.1 mm and less than about 1 mm.
K. A method of cleaning a mammal, preferably a household pet, more preferably a dog, the method comprising the steps of: (a) providing a wet wipe according to any one of the preceding paragraphs; (b) wiping the mammal, preferably the household pet, more preferably the dog, with the wet wipe according to any one of the preceding paragraphs.
L. A kit comprising: (a) a liquid composition comprising: (i) about 0.01% to about 0.2% by weight of the composition of a cationic polymer, where the cationic polymer has a calculated cationic charge density of about 0.1 to about 4.0, preferably about 0.4 to about 3.2, more preferably about 0.75 to about 2.5, and a weight average molecular weight of about 10,000 g/mol to about 1,750,000 g/mol, preferably about 10,000 g/mol to about 1,600,000 g/mol, more preferably about 10,000 g/mol to about 1,500,000 g/mol; (ii) about 80% to about 99.5% by weight of the composition of water; where the composition has a surface tension of about 30 mN/m to about 60 mN/m and a pH of about 3 to about 6; and (b) a non-pre-moistened non-woven substrate comprising: (i) about 0% to about 90%, preferably about 1% to about 85%, more preferably about 5% to about 55%, even more preferably about 10% to about 35%, by weight of the substrate of synthetic fibers; (ii) about 10% to about 100%, preferably about 10% to about 65%, more preferably about 14% to about 45%, by weight of the substrate of natural fibers; where the non-woven substrate has a basis weight from about 40 g/m² to about 100 g/m², preferably about 40 g/m² to about 85 g/m², more preferably about 45 g/m² to about 80 g/m².
M. A method of cleaning a mammal, preferably a household pet, more preferably a dog, the method comprising the steps of dispensing the liquid composition according to paragraph L onto the mammal, preferably the household pet, more preferably the dog, and wiping the mammal, preferably the household pet, more preferably the dog, with the non-pre-moistened non-woven substrate of paragraph M.

### Test Methods

All test methods are carried out in an environment 23±2° C. and 50±5% relative humidity environment, unless otherwise specified.

### Tile Test Method

Procedure for measuring cleaning performance using a 30.5 cm×30.5 cm smooth black tile (The Tile Shop, Item #681473, USA). First, the tile is cleaned by rinsing with water three times and wiping the tile dry with a clean paper towel between each rinse. Then, the tile is rinsed a final time with isopropanol and wiped dry with a paper towel. Clean tiles are photographed and analyzed in ImageJ to serve as a control. A standardized soil solution is prepared by mixing white cosmetic clay (kaolin) (Mountainroseherbs.com, USA) with water at a 3:4 weight ratio of clay to water. 2.5 mL of this standardized soil is added to the center of the tile. Starting at one corner of the tile and making W-motions, a wipe is used to clean the entire surface. Then, the wipe is folded in half and the clean side of the wipe is used to clean the entire surface again, using W-motions. The folding and cleaning steps are repeated a third time and the tile is then allowed to dry.

The tile is photographed in a light box and imported into the ImageJ application (ImageJ-PG, Version 1.53u.57, Oct. 28, 2022 USA). Using ImageJ, information about the color of each pixel in the tile image is generated on a scale from 0 (pure black) to 255 (pure white). The mean value for the whole tile is recorded. This procedure is done is triplicate and the three mean values are exported to JMP (JMP Pro 16.1.0, SAS Institute Inc., USA), where Tukey's Test for significant difference is run. The significance level for all tests was set at α=0.05.

### Black Glove Test Method

Based on an observation that large amounts of the standardized soil described above was transferring through the substrate to the hand of the individual performing the Tile Test, the Black Glove Test was developed to evaluate the transfer of soil through the substrate. The procedure for carrying out the Black Glove Test involves the same steps as described above for the Tile Test, except that, after applying 2.5 mL of standardized soil to the tile, the tester places his or her hands in a new pair of black nitrile gloves (Rip Resistant Industrial Gloves, Medline Industries, USA). The cleaning procedure is carried out as described above for the Tile Test. After the procedure is completed, the tester evaluates the gloves for the presence of soil (on the portions of the gloves that contact the wipe during cleaning). The presence or absence of standardized soil is documented as “pass” or “fail,” respectively.

### Surface Tension Measurement Method

Procedure for Measuring Surface Tension Using Kruss 100 Tensiometer: surface tension is measured using a Kruss Model 100 Tensiometer (Kruss GMBH, Germany) or equivalent and Advance software. A Wilhelmy platinum probe PL01 is used with a wetting length of 40.2 mm. Both surface tension (mN/m) and temperature (° C.) are recorded.

A liquid composition is tested (volume) in 50 mL beaker. Samples are equilibrated to room temperature (21-24° C.) and then tested in duplicate. Water controls (±1 mN/m of expected value) are run before and after each composition to ensure the platinum probe is thoroughly clean. Expected Value (water)=72.86 mN/m−(20° C. —Temp.) (−0.1514 mN/m/° C.).

### Liquid Composition Expression Method

Liquid composition is expressed from wet wipes for further analysis using the Liquid Composition Expression Method. According to this method, liquid composition is extracted from a package of marketed wet wipes.

At ambient temperature, a package of marketed wet wipes is opened and the wipes are removed. The wipes are folded in half to form a stack with approximate footprint dimensions 0.10 m×0.13 m. The stack of wipes is then placed between the two plates of a Healthy Express Extra Firm Tofu Press (5 star North, available on Amazon.com, USA) and the press with the stack of wipes is then placed in a suitable container to collect the expressed liquid. The screws of the tofu press are tightened to a finger-tight level and the wipes are allowed to sit for at least one minute and until no more fluid drains freely from the wipes. The expressed liquid is then placed in a suitable storage container, sealed, and stored at ambient temperature until further analysis.

### Water Content Test Method

In the Water Content Test Method, a portion of expressed liquid composition is placed in an oven to facilitate evaporation, and the remaining unevaporated mass is measured. From this, the water content of the starting expressed liquid composition is calculated.

The Liquid Composition Expression Method is used to express liquid composition, from which a 5.0±0.1 g aliquot is taken and placed in a 70-mm diameter aluminum weighing boat (such as VWR part number 25433-089, VWR International, Radmor, PA, USA, or equivalent), and the initial mass of the liquid composition aliquot is determined to at least the nearest 0.001 g. Immediately following weighing, the weighing boat containing the liquid composition is placed in an oven held at 100° C. for 12±1 hour, at which point the boat containing the unevaporated material remaining from the aliquot is removed, and the mass of unevaporated material is determined to the nearest 0.001 g. The quotient of the unevaporated mass remaining from the liquid composition aliquot to the initial mass of the liquid composition aliquot, expressed as a percent to the nearest tenth of a percent, is defined as the Liquid Composition Percent Solids Parameter. The Liquid Composition Percent Solids Parameter is subtracted from 100.0%, and the resulting difference is defined as the Liquid Composition Percent Water Parameter.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this present disclosure.

What is claimed is:

1. A wet wipe comprising:
a. a liquid composition comprising:
i. about 0.01% to about 0.2% by weight of the composition of a cationic polymer, wherein the cationic polymer has a calculated cationic charge density of about 0.1 to about 4.0 and a weight average molecular weight of about 10,000 g/mol to about 1,750,000 g/mol;
ii. from about 0.001 wt. % to about 5 wt. % by weight of the composition of a cleaning solvent selected from the group consisting of C3-C10 diols; and
iii. about 95% to about 99.5% by weight of the composition of water;
wherein the composition has a pH of about 3 to about 6; and
b. a non-woven substrate comprising:
i. about 1% to about 90% by weight of the substrate of synthetic fibers;
ii. about 10% to about 100% by weight of the substrate of natural fibers;
wherein the non-woven substrate has a basis weight from about 40 g/m² to about 100 g/m²;
wherein the loading ratio of liquid composition to non-woven substrate is about 2.0 g/g to about 6.0 g/g.

2. The wet wipe of claim 1, wherein the cleaning solvent is a C6-C8 diol.

3. The wet wipe of claim 1, wherein the cleaning solvent is a mixture of 1,2-octanediol and 1,2-hexanediol.

4. The wet wipe of claim 1, wherein the non-woven substrate has an absorption capacity of about 9 g/g to about 11 g/g.

5. The wet wipe of claim 1, wherein the substrate is free of polyamides.

6. The wet wipe of claim 1, wherein the natural fibers comprise cellulose fibers.

7. The wet wipe of claim 6, wherein the cellulose fibers comprises viscose fibers.

8. The wet wipe of claim 1, wherein the liquid composition is substantially free of ionic surfactants.

9. The wet wipe of claim 8, wherein the liquid composition is substantially free of anionic surfactants.

10. A wet wipe comprising:
a. a liquid composition comprising:
i. about 0.01% to about 0.2% by weight of the composition of a cationic polymer, wherein the cationic polymer has a calculated cationic charge density of about 0.1 to about 4.0 and a weight average molecular weight of about 10,000 g/mol to about 1,750,000 g/mol; and
ii. about 80% to about 99.5% by weight of the composition of water;
wherein the composition has a surface tension of about 30 mN/m to about 60 mN/m and a pH of about 3 to about 6; and
b. a non-woven substrate comprising:
i. about 1% to about 90% by weight of the substrate of synthetic fibers;
ii. about 10% to about 100% by weight of the substrate of natural fibers;
wherein the non-woven substrate has a basis weight from about 40 g/m² to about 100 g/m²;
wherein the loading ratio of liquid composition to non-woven substrate is about 2.0 g/g to about 6.0 g/g.

11. The wet wipe of claim 10, wherein the non-woven substrate has an absorption capacity of about 9 g/g to about 11 g/g.

12. The wet wipe of claim 10, wherein the substrate is free of polyamides.

13. The wet wipe of claim 10, wherein the natural fibers comprise cellulose fibers.

14. The wet wipe of claim 13, wherein the cellulose fibers comprises viscose fibers.

15. The wet wipe of claim 10, wherein the liquid composition comprises about 0.05 wt. % to about 9.0 wt. % of any emulsifier.

16. The wet wipe of claim 10, wherein the area of the wet wipe is about 100 cm² to about 1000 cm².

17. The wet wipe of claim 10, wherein the wet wipe is disposable.

18. The wet wipe of claim 10, wherein a caliper of the wet wipe is greater than about 0.1 mm and less than about 1 mm.

19. A kit comprising:
a. a liquid composition comprising:
i. about 0.01% to about 0.2% by weight of the composition of a cationic polymer, wherein the cationic polymer has a calculated cationic charge density of about 0.1 to about 4.0 and a weight average molecular weight of about 10,000 g/mol to about 1,750,000 g/mol; and
ii. about 80% to about 99.5% by weight of the composition of water;
wherein the composition has a surface tension of about 30 mN/m to about 60 mN/m and a pH of about 3 to about 6; and
b. a non-pre-moistened non-woven substrate comprising:
i. about 1% to about 90% by weight of the substrate of synthetic fibers;
ii. about 10% to about 100% by weight of the substrate of natural fibers;
wherein the non-woven substrate has a basis weight from about 40 g/m² to about 100 g/m².

20. A method of cleaning a mammal, the method comprising the steps of:
a. providing a wet wipe of claim 1; and
b. wiping the mammal with the wet wipe of claim 1.
